SEQUENCE LISTING (20 sequences)

SEQ ID NO: 1 - DNA, 2128 bp, Homo sapiens
ctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgacaaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaaaaaaaaaaaaaaaaaaaaaaa

SEQ ID NO: 2 - DNA, 20 bp, Artificial Sequence
  Primer
cgagatccctccaaaatcaa

SEQ ID NO: 3 - DNA, 20 bp, Artificial Sequence
  Primer
atccacagtcttctgggtgg

SEQ ID NO: 4 - DNA, 20 bp, Artificial Sequence
  Primer
attgtgggaggctgggagtg

SEQ ID NO: 5 - DNA, 20 bp, Artificial Sequence
  Primer
gtcaccttctgagggtgaac

SEQ ID NO: 6 - DNA, 20 bp, Artificial Sequence
  Primer
atgtgagccaaatgcaatgt

SEQ ID NO: 7 - DNA, 25 bp, Artificial Sequence
  Primer
cattaccctgctatatttgtcaaga

SEQ ID NO: 8 - DNA, 24 bp, Artificial Sequence
  Primer
catccatgacaactttggtatcgt

SEQ ID NO: 9 - DNA, 19 bp, Artificial Sequence
  Primer
ccatcacgccacagtttcc

SEQ ID NO: 10 - DNA, 24 bp, Artificial Sequence
  Primer
ggttgtctggaggacttcaataca

SEQ ID NO: 11 - DNA, 20 bp, Artificial Sequence
  Primer
gagggagggtcttcctttgg

SEQ ID NO: 12 - DNA, 25 bp, Artificial Sequence
  Primer
aaatcatggaaaataagggaatcct

SEQ ID NO: 13 - DNA, 21 bp, Artificial Sequence
  Primer
ccaagaagcacattgcatttg

SEQ ID NO: 14 - DNA, 27 bp, Artificial Sequence
  Probe
aaggactcatgaccacagtccatgcca

SEQ ID NO: 15 - DNA, 29 bp, Artificial Sequence
  Probe
actgaccccctggaagctgattcactatg

SEQ ID NO: 16 - DNA, 36 bp, Artificial Sequence
  Probe
agaaacaatcacagaccacatgagactaaggagaca

SEQ ID NO: 17 - DNA, 32 bp, Artificial Sequence
  Primer
gttgttctcgagcgcattacacaccgagtagg

SEQ ID NO: 18 - DNA, 32 bp, Artificial Sequence
  Primer
caacaagaattcgcaactacagccaagggttc

SEQ ID NO: 19 - DNA, 32 bp, Artificial Sequence
  Primer
caacaagaattccgcattacacaccgagtagc

SEQ ID NO: 20 - DNA, 32 bp, Artificial Sequence
  Primer
gttgttctcgaggcaactacagccaagggttc

(19) United States
(12) Patent Application Publication
(10) Pub. No.: US 2003/0027178 A1
(43) Pub. Date: Feb. 6, 2003

(54) Methods and kits for determining a cancer diagnosis and prognosis

(76) Inventors: George Vasmatzis, Rochester, MN (US); Farhad Kosari, Rochester, MN (US); Yan Asmann, Rochester, MN (US); John Cheville, Stewartville, MN (US)

Correspondence Address: MARK S. ELLINGER, PH.D., Fish & Richardson P.C., P.A., Suite 3300, 60 South Sixth Street, Minneapolis, MN, 55402, US

(21) Appl. No.: 10/099,570

(22) Filed: Mar. 15, 2002

Related U.S. Application Data
(60) Provisional application No. 60/276,523, filed on Mar. 16, 2001.

(51) Int. Cl. C12Q 1/68; G01N 33/574

(52) U.S. Cl. 435/6; 435/7.23

(57) ABSTRACT

The invention provides methods and materials for determining cancer diagnosis and prognosis based on examining the levels of CRISP-3 RNA and polypeptides in biological samples from a patient. The invention also provides kits that can be used to determine a cancer diagnosis or prognosis. The kits contain CRISP-3 antibodies, CRISP-3 nucleic acids, PSA antibodies, PSA nucleic acids, or any combinations of these.

### CROSS REFERENCE TO RELATED APPLICATIONS

This application claims priority from U.S. Provisional Application Serial No. 60/276,523 filed on Mar. 16, 2001.

### BACKGROUND

1. Technical Field

The invention relates to methods and materials useful for determining cancer diagnosis and prognosis.

2. Background Information

Prostate cancer is the second leading cause of cancer death among American men. Prostate cancer kills close to 40,000 men per year in the United States. Early diagnosis of prostate cancer, however, has almost doubled the number of patients that survive for at least 5 years.

Methods currently available for diagnosing prostate cancer include digital rectal exam, transrectal ultrasonography, intravenous pyelogram, cystoscopy, and blood and urine tests for levels of prostate specific antigen (PSA) or prostatic acid phosphatase (PAP). A recurrent issue with the use of some of these methods is accessibility. A growing tumor that is not adjacent to the rectum can be difficult to detect by digital rectal examination. Similarly, not all epithelial tissues in the prostate are accessible to ultrasound, pyelogram, and cystoscopy examinations. Blood and urine tests for PSA have been widely used in clinical diagnosis of prostate cancer. Conditions such as benign prostatic hyperplasia (BPH), prostatitis, and prostate intraepithielial neoplasia (PIN), however, can result in an elevated level of PSA in the blood and urine. Therefore, accurate and specific methods for prostate cancer diagnosis and prognosis are needed. Similarly, improved methods for determining a diagnosis or prognosis of other cancer, such as pancreas cancer, are desirable.

### SUMMARY

The invention provides methods and materials related to the diagnosis and prognosis of cancer. More specifically, the invention relates to the diagnosis and prognosis of cancer in tissues that produce the CRISP-3 polypeptide; some examples include cancer of the prostate and pancreas. The invention is based on the discovery that elevated levels of CRISP-3 RNA and CRISP-3 polypeptides are produced in malignant cells. Therefore, by examining the level of CRISP-3 RNA or CRISP-3 polypeptides in a biological sample, a cancer diagnosis or prognosis is determined.

In general, the invention features a method for determining whether or not a patient has cancer by detecting CRISP-3 expression in a biological sample. CRISP-3 expression can be examined quantitatively by determining the levels of CRISP-3 RNA and polypeptide. The invention is used to diagnose cancer in a mammal, for example, a human. Cancers that can be diagnosed by the invention include prostate cancer, pancreas cancer, salivary gland cancer, lung cancer, ovarian cancer, thymus cancer, hematological cancer, and descending colon cancer. The biological sample typically is a cell sample or a body fluid. Cell samples can be biopsy specimens, for example prostate epithelial cell specimens and pancreas cell specimens, as well as metastatic tumor specimens. Cell samples also can include cells isolated from a body fluid, for example blood cells. The body fluid can be any organ fluid or secretion, for example, blood, bone marrow, urine, semen, saliva, vaginal secretions, cerebrospinal fluid, and pancreatic fluid.

In another embodiment, the invention provides a method for identifying a cancer cell in a cell sample by detecting CRISP-3 RNA or CRISP-3 polypeptide and measuring the levels of CRISP-3 RNA or polypeptide in the cell sample. In this embodiment, the cell sample typically is a biopsy specimen that contains malignant (cancerous) and normal cells.

In another embodiment, the invention also provides a method for distinguishing BPH, PIN, and various stages of cancer by determining the level of CRISP-3 RNA or CRISP-3 polypeptide and correlating the level of CRISP-3 RNA or CRISP-3 polypeptide with BPH, PIN (e.g., high grade PIN), or various stages of cancer.

The invention also provides a method for determining a prognosis for a cancer condition by determining the level CRISP-3 RNA or CRISP-3 polypeptide in a biological sample and correlating the level of CRISP-3 RNA or CRISP-3 polypeptide with a cancer prognosis.

In another embodiment, the invention features a CRISP-3 antibody that binds specifically to a CRISP-3 polypeptide, and methods to make the CRISP-3 antibody. To make a CRISP-3 antibody, a non-human animal is immunized with a CRISP-3 polypeptide or a nucleic acid molecule that allows for expression of a CRISP-3 polypeptide in the non-human animal. The serum of the immunized animal can be used directly, or a CRISP-3 antibody can be isolated from the serum of the immunized animal. The non-human animal can be, without limitation, a rabbit, a chicken, a mouse, a guinea pig, a rat, a sheep, or a goat.

Methods to make a CRISP-3 antibody also include generating a CRISP-3 antibody-producing hybridoma cell. The CRISP-3 antibody-producing hybridoma cell is isolated from an animal immunized with a CRISP-3 polypeptide or a CRISP-3 expressing nucleic acid. The hybridoma cell is cultured and CRISP-3 monoclonal antibody is isolated from the culture supernatant.

The invention also features a method for determining the presence or absence of a cancer cell in a biological sample. The method involves exposing the biological sample to a CRISP-3 antibody and detecting the presence or absence of specific hybridization of the CRISP-3 antibody with the cancer cell.

In another embodiment, the invention provides an isolated nucleic acid of 800 bases or less that hybridizes under high stringency conditions to a nucleic acid consisting of nucleotides 650 to 1450 of SEQ ID NO: 1. In another embodiment, the invention provides an isolated nucleic acid of 800 bases or less that hybridizes under high stringency conditions to the complement of a nucleic acid consisting of nucleotides 650 to 1450 of SEQ ID NO: 1.

In another embodiment, the invention provides an isolated nucleic acid that hybridizes under high stringency conditions to the 3′ untranslated region of human CRISP-3 RNA but not to the 3′ untranslated regions of human CRISP-1 and CRISP-2 RNA. The isolated nucleic acid can have sequences set out in SEQ ID. NO: 6, SEQ ID. NO: 7, SEQ ID. NO: 12, SEQ ID. NO: 13, SEQ ID. NO: 16, SEQ ID. NO: 17, or SEQ ID. NO: 18. The isolated nucleic acid also can be labeled.

In another embodiment, the invention features a method for determining the presence or absence of a cancer cell in a biological sample by exposing the biological sample to an isolated nucleic acid described above, and detecting the presence or absence of specific hybridization of the isolated nucleic acid with a cancer cell. The biological sample can be human tissue in the form of a histological specimen. The cancer cell can be, without limitation, a prostate cell or a pancreas cell. The cancer cell also can be a metastastic cancer cell.

In another embodiment, the invention provides a method for determining the presence of a cancer cell in a biological sample using a nucleic amplification method. Nucleic acid amplification methods include, without limitation, reverse transcription-polymerase chain reation (RT-PCR), transcription-based amplification system (TAS), self-sustained sequence replication (3SR), strand displacement amplification (SDA), ligase chain reaction (LCR), repair chain reaction (RCR), and Boomerang DNA amplification (BDA). Typically, RNA is isolated from cells and subjected to reverse transcription; this is followed by DNA or RNA amplification. Alternatively, a direct amplification of the RNA can be performed. CRISP-3 primers provided by the invention can be used. The presence of CRISP-3 amplification products indicates that the biological sample contains malignant cells.

The invention also features kits useful for determining a cancer diagnosis or prognosis. Kits can include CRISP-3 antibodies, combinations of CRISP-3 and PSA antibodies, nucleic acids that hybridize to CRISP-3 nucleic acids, combinations of nucleic acids that hybridize to CRISP-3 and PSA nucleic acids, or any combination of the above.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. An isolated nucleic acid includes, without limitation, a recombinant DNA that exists as a separate molecule, for example, a cDNA or a genomic DNA fragment produced by RT-PCR, PCR, or restriction endonuclease treatment as well as recombinant DNA that is incorporated into a vector, an autonomously replicating plasmid, or a virus. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, or a virus. In addition, a non-naturally-occurring nucleic acid can include a nucleic acid molecule that is part of a hybrid or fusion nucleic acid sequence.

The term “isolated” or “purified” as used herein with reference to polypeptide (e.g., an antibody), or biologically active portion thereof refers to polypeptide or biologically active portion thereof that is substantially free of cellular material or other contaminating polypeptides from the cell or tissue source from which the polypeptide is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of protein having less than about 30%, 20%, 10%, or 5% (by dry weight) of heterologous polypeptide (also referred to herein as a “contaminating polypeptide”). When the polypeptide or biologically active portion thereof is recombinantly produced, it is also preferably substantially free of culture medium, i.e., culture medium represents less than about 20%, 10%, 5% of the volume of the polypeptide preparation. When the polypeptide is produced by chemical synthesis, it is preferably substantially free of chemical precursors or other chemicals, i.e., it is separated from chemical precursors or other chemicals that are involved in the synthesis of the polypeptide. Accordingly such preparations of the polypeptide have less than about 30%, 20%, 10%, 5% (by dry weight) of chemical precursors or compounds other than the polypeptide of interest.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1a is the nucleotide sequence of CRISP-3. (SEQ ID NO: 1)

FIG. 1b is an illustration of the regions of overlap between the CRISP-3 cDNA sequence and forty CRISP-3 expressed sequence tag (EST) sequences found in a human EST database. Each dotted line represents the full-length CRISP-3 cDNA sequence. Each dashed arrowhead line represents an EST sequence and marks the region of overlap with the CRISP-3 cDNA sequence. Each “x” indicates a nucleotide mismatch between the EST sequence and the CRISP-3 cDNA sequence. An EST identification is shown on the left of each line. A library identifier, shown on the right of each line, indicates the source of the EST. In the library identifiers: CM indicates a microdissected cancer library; NB indicates a normal bulk library; CB indicates a cancel bulk library; 575 indicates a cDNA library constructed from microdissected prostate cancer tissues; 919 indicates a library constructed from a needle biopsy of a metastatic prostate bone lesion; 1410 indicates a library constructed from bulk prostate tissues; 1076 indicates a library constructed from microdissected lung cancer tissues; and 2460 indicates a library constructed from whole blood CML.

FIG. 2 is a graphical illustration of the representation of PSA, Hk2, GAPDH, and CRISP-3 genes in EST prostate libraries as determined by electronic profiling.

FIG. 3 is a graphical illustration of real time RT-PCR results for amplification of (GAPDH, PSA, and CRISP-3 RNA from normal and cancer cells. Dotted lines represent results obtained for normal tissues and solid lines represent results for cancer tissues.

FIG. 4 is a comparison of Δ(CT) values for real time RT-PCR amplification of GAPDH, PSA, and CRISP-3 RNA in six prostate cancer cases.

FIG. 5 is a comparison of the amounts of PSA and CRISP-3 produced by cancerous and benign prostate tissues.

FIG. 6a is a graph that summarizes the ΔΔ(C_{T}) values determined for five benign/GP3 pairs and three PIN/GP3 pairs.

FIG. 6b is a graph that summarizes the ΔΔ(C_{T}) values determined when bulk samples of moderately differentiated or poorly differentiated prostate adenocarcinoma were compared with benign prostate cells.

### DETAILED DESCRIPTION

The invention provides methods and materials related to the diagnosis and prognosis of cancer in a mammal such as a human. Diagnostic and prognostic methods and materials provided by the invention are based on examining CRISP-3 expression.

CRISPs, cysteine-rich secretory proteins, are highly conserved proteins and derive their names from a cluster of cysteine residues in the C-terminal portion that form a discrete, compact domain (Eberspaecher et al. (1995) Mol Reprod Dev 42:57-72). The first member of the CRISP family of proteins was characterized in the rat epididymis as protein DE (Cameo and Blaquier (1976) J Endocrine 69:47-55) and acidic epididymal glycoprotein (AEG) (Lea et al. In: Endocrinol approach to male contraception, Copenhagen, Scriptor, (1978) pg. 592-607). The corresponding cDNA was subsequently cloned from the same organ (Brooks et al (1986) Eur J Biochem 161:13-18). The mouse and human orthologues of protein DA/AEG were named CRISP-1 (Haendler et al. (1993) Eur J Biochem 250:440-446 and Kratzschmar et al. (1996) Eur J Biochem 236: 827-36). In addition, two related forms, CRISP-2 (also called Tpx-1) and CRISP-3, were identified in the testis and salivary gland, respectively (Kasahara et al. (1989) Genomics 5:527-34; Mizuki and Kasahara (1992) Mol Cell Endocrinol 89:25-32; and Haendler et al. (1993) Eur J Biohem 250:440-446). In humans, CRISP-3 mRNA is found predominantly in the salivary gland, pancreas, and prostate. CRISP-3 mRNA is found in less abundance in human epididymis, ovary, thymus, and colon (Kratzschmar et al. (1996) Eur J Biochem 236: 827-36). CRISP-3 also has been detected in two leukocytic cell types, murine pre-B cells (Pfisterer et al. (1996) Mol Cell Biol 16: 6160-8) and human neutropils (Kjeldsen et al. (1996) FEBS Lett 380: 246-50).

The expression of CRISPs on mucosal epithelial surfaces, by glands with an exocrine function, and by cells of the immune system has led to the hypothesis that CRISPs function in nonspecific host defense similar to defensins (Kagan et al. (1994) Toxicology 87:131-49) and magainins (Berkowitz et al. (1990) Biochem Pharmacol 39: 625-9). This hypothesis is supported by the similarity in amino acid sequences of CRISPs and the PR-1 group of plant pathogenesis-related proteins (Fritig et al. (1998) Curr Opin Immunol 10:16-22). Supportive experimental evidence, however, is lacking. In addition, CRISP-1 was reported to be involved in the late stages of sperm maturation (Eberspaecher et al. (1995) Mol Reprod Dev 42:157-72). Human CRISP-3 shares about 67% and 42% homology with CRISP-1 and CRISP-2, respectively. Although CRISP-3 has been considered to play a role in the fertilization process, little is known about the function of CRISP-3.

The invention is based on the discovery that CRISP-3 expression is elevated in some cancer cells, for example, cancerous prostate epithelial cells. Therefore, by examining CRISP-3 expression in an appropriate biological sample, cancers such as prostate cancer and pancreas cancer can be diagnosed. Methods for examining CRISP-3 expression can be qualitative or quantitative. CRISP-3 expression can be examined by detecting CRISP-3 RNA (e.g. mRNA), polypeptide, or both RNA and polypeptide. CRISP-3 expression also can be examined by measuring the levels of CRISP-3 RNA (e.g. mRNA), polypeptide, or both RNA and polypeptide in a biological sample. The invention provides materials (CRISP-3 antibodies and CRISP-3 nucleic acids, e.g. primers and hybridization probes) that can be used to examine the levels of CRISP-3 RNA and polypeptide in a biological sample. The invention also provides kits for detecting CRISP-3 RNA and polypeptide, for determining the levels of CRISP-3 RNA and polypeptide, and for determining the levels of CRISP-3 RNA and polypeptides in combination with the levels of PSA RNA and polypeptide in a biological sample. Various aspects of the invention are described in further detail in the following sections.

1. Preparation of Purified CRISP-3 Polypeptides

CRISP-3 polypeptides can be obtained from a number of sources. For example, CRISP-3 polypeptides can be obtained from cells that naturally express CRISP-3 polypeptides. These include, without limitation, cells from the prostate, pancreas, salivary gland, lung, ovary, thymus, and descending colon. CRISP-3 polypeptides also can be obtained from cells engineered to produce CRISP-3 polypeptides by recombinant DNA technology. In recombinant DNA technology, pieces of nucleic acid molecules from different organisms or different sources are ligated together to form a recombinant nucleic acid molecule. A cell engineered to produce a CRISP-3 polypeptide typically contains a recombinant nucleic acid expression vector, i.e., a CRISP-3 expression vector, having a CRISP-3 polypeptide-coding region.

CRISP-3 expression vectors typically have CRISP-3 polypeptide-coding regions as well as expression control sequences needed for expression of the CRISP-3 polypeptide-coding regions. Expression control sequences include promoters, ribosome binding sites, enhancers, and any other nucleic acid elements required for transcription and translation of polypeptide-coding regions. See Bittner et al. (1997) Methods in Enzymol. 153:516-544. Expression control sequences are operably linked to polypeptide-coding regions; i.e., they are linked in a manner that allows for transcription and translation of the polypeptide-coding region. The CRISP-3 expression vector also can have additional nucleic acid sequences positioned 5′, 3′, or 5′ and 3′ of the CRISP-3 polypeptide-coding region. The additional nucleic acid sequence can encode a polypeptide domain that is fused to the CRISP-3 polypeptide when the CRISP-3 polypeptide-coding region and the additional nucleic acid sequence are expressed. The polypeptide domain that is fused to the CRISP-3 polypeptide can, for example, aid in the purification of the CRISP-3 polypeptide. Examples of polypeptide domains useful for purification include, without limitation, glutathione S transferase, maltose E binding protein, protein A, and a poly-histidine tag.

The CRISP-3 expression vector can be introduced into a host cell by any standard method for introducing nucleic acids into a cell. These methods can include electroporation, transfection, transduction, conjugation, and any bacterial transformation method known in the art. The host cell can be any eukaryotic or prokaryotic cell, for example, an E. coli cell, a yeast cell, or any mammalian cell.

The host cell can be cultured under any condition that allows for expression of the CRISP-3 polypeptide-coding region. Since CRISP-3 has a signal sequence in the first 20 amino acids, it is a secretory polypeptide and typically is purified from the culture supernatant of the host cell culture. Any standard protein purification method can be used for purifying CRISP-3 polypeptides. Steps in the purification procedure can include, without limitation, ammonium sulfate precipitation, ion exchange chromatography, and size exclusion chromatography. CRISP-3 polypeptides also can be purified by affinity chromatography or immuno-precipitation.

2. Anti-CRISP-3 Antibody

One aspect of the invention pertains to an “anti-CRISP-3 antibody,” i.e., an antibody directed to a CRISP-3 polypeptide. The term “CRISP-3 polypeptide” refers to the entire CRISP-3 polypeptide, as well as any immunogenic fragment of the CRISP-3 polypeptide that does not elicit an antibody response that is cross-reactive with another member of the CRISP family of proteins. The term “anti-CRISP-3 antibody” as used herein refers to any immunoglobulin molecule that binds specifically to a CRISP-3 polypeptide.

A molecule that is said to “bind specifically” or “hybridize specifically” to a second molecule in a biological sample will bind or hybridize to that second molecule without substantially binding or hybridizing to other molecules present in the same biological sample. For example, an anti-CRISP-3 antibody is said to bind specifically to a CRISP-3 polypeptide if it binds to the CRISP-3 polypeptide but will not bind to any other member of the CRISP family that is present in the same biological sample.

The term “anti-CRISP-3 antibody” includes a whole antibody as well as an immunologically active fragment of an immunoglobulin molecule that binds specifically to the CRISP-3 polypeptide. An immunologically active fragment of an immunoglobulin molecule has the same antigen-binding site and therefore the same antigen specificity as the complete immunoglobulin molecule. Examples of immunologically active fragments encompassed by the invention include F(ab) and F(ab′)2 fragments that recognize the CRISP-3 polypeptide.

The term “anti-CRISP-3 antibody” includes monoclonal, polyclonal, and recombinant antibodies that bind specifically with the CRISP-3 polypeptide. A monoclonal antibody is a homogenous population of antibody molecules. All antibody molecules of the monoclonal antibody population have the same antigen-binding site and bind the same epitope on an antigen. In contrast, a polyclonal antibody is a heterogeneous population of antibody molecules. Antibody molecules of the polyclonal antibody population recognize different epitopes of the same antigen. A recombinant antibody is a non-naturally occurring antibody that is encoded by a recombinant nucleic acid molecule. Typically, a non-naturally occurring antibody has portions that come from different organisms or different sources. One example of a non-naturally occurring antibody is a chimeric humanized antibody that consists of a human portion and a non-human portion. An anti-CRISP-3 antibody includes any recombinant antibody that recognizes the CRISP-3 polypeptide.

Anti-CRISP-3 polyclonal or monoclonal antibody can be produced using various methods. One method involves immunizing a non-human host animal with purified CRISP-3 polypeptides. The non-human host animal also can be immunized with a recombinant nucleic acid molecule that has a CRISP-3 polypeptide-coding region. See Chowdhury et al. (2001) J Immunol Methods 249:147-154 and Boyle et al. (1997) Proc Natl Acad Sci U.S.A 94:14626-31. This recombinant nucleic acid molecule also has expression control sequences such as a promoter, a ribosome-binding site, and any other control sequences necessary for transcription and translation of the CRISP-3 polypeptide-coding region. These expression control sequences are operably linked to the CRISP-3 polypeptide-coding region and allow for CRISP-3 polypeptide expression.

The non-human host animal that is immunized for antibody production can be, without limitation, a rabbit, a chicken, a mouse, a guinea pig, a rat, a sheep, or a goat. Blood serum from the immunized non-human host animal is used as a source of anti-CRISP-3 polyclonal antibody. To obtain an anti-CRISP-3 polyclonal antibody from the blood serum of an immunized host animal, any standard method can be used. Typically, an anti-CRISP-3 polyclonal antibody is isolated from blood serum using protein A chromatography.

Anti-CRISP-3 monoclonal antibody can be obtained using B-lymphocytes isolated from an immunized human host animal. Typically, antibody-producing B-lymphocytes are isolated from the spleen of the immunized host animal at a time after immunization when serum antibody titer is highest. Serum anti-CRISP-3 antibody titer can be determined using any standard method. For example, enzyme linked immunosorbent assay (ELISA) can be used to determine the titer of an anti-CRISP-3 antibody in a sample. In ELISA, the CRISP-3 polypeptide typically is immobilized on a surface. The immobilized polypeptide is exposed to serum containing anti-CRISP-3 antibody under conditions that allow for specific binding of the antibody to the polypeptide. The bound anti-CRISP-3 antibody can be detected with a second antibody that is conjugated with a readily detectable marker such as an enzyme, a fluorescent molecule, or a radioactive molecule. Once isolated, B-lymphocytes are fused with myeloma cells to generate hybridoma cells. Standard hybridoma fusion methods are described in Kohler and Milstein (1975) Nature 256:495-497 and 1Kozbor et al. (1983) Immunol Today 4:72. Hybridoma cells are cultured singly so that each culture results from the growth of one hybridoma cell. An anti-CRISP-3 antibody-producing hybridoma cell can be identified by screening culture supernatants of different hybridoma cell cultures for an antibody that binds to the CRISP-3 polypeptide. Anti-CRISP-3 antibody in the supernatant can be identified using ELISA as described above.

An anti-CRISP-3 monoclonal antibody also can be obtained by using commercially available kits that aid in preparing and screening antibody phage display libraries. An antibody phage display library is a library of recombinant combinatorial immunoglobulin molecules. Examples of kits that can be used to prepare and screen antibody phage display libraries include the Recombinant Phage Antibody System (Pharmacia) and SurfZAP Phage Display Kit (Stratagene). To identify an anti-CRISP-3 monoclonal antibody in the library, the library is screened using the CRISP-3 polypeptide.

A recombinant chimeric humanized antibody, an immunologically active immunoglobulin fragment, and a single chain antibody specific for the CRISP-3 polypeptide can be prepared using known techniques such as those described in Better et al. (1988) Science 240:1041-1043, Jones et al. (1986) Nature 321:552-525, and U.S. Pat. Nos. 4,946,778 and 4,704,692. A chimeric humanized antibody can be produced by ligating a portion of a mouse antibody coding sequence specific for the antigen of interest with a portion of a human antibody coding sequence. An immunologically active immunoglobulin fragment such as a F(ab′)2 fragment can be generated by digestion of an antibody with pepsin while a Fab fragment can be obtained by reduction of the disulfide bridges of the F(ab′)2 antibody fragment. A single chain antibody can be formed by linking the heavy and light chains of an immunoglobulin molecule together with an amino acid bridge.

3. Antibody-based Assays

An anti-CRISP-3 antibody can be used in various assays to detect CRISP-3 polypeptides in a biological sample. Any method involving the formation and detection of a CRISP-3 polypeptide-anti-CRISP-3 antibody complex can be used to practice the invention. For example, a CRISP-3 polypeptide in a biological sample can be detected using conventional western hybridization. In western hybridization, polypeptides in the sample are separated in a gel matrix by electrophoresis. The separated polypeptides are then transferred to a membrane and the membrane is contacted with an anti-CRISP-3 antibody under conditions that allow for formation of an antigen-antibody complex. Typically, the CRISP-3 polypeptide-antibody complex can be detected using a labeled second antibody that hybridizes to the first anti-CRISP-3 antibody. CRISP-3 polypeptides in any biological fluid, for example, sera, semen, or urine, can be detected in this way.

Western hybridization also can be used to analyze CRISP-3 polypeptides in tissues and particular cell types. Particular cell types can be isolated by microdissection. Tissues and cells can be solubilized with a lysis buffer or homogenized with a homogenizer prior to electrophoretic analysis. After solubilization or homogenization, polypeptides can be separated by electrophoresis and detected using western hybridization.

CRISP-3 polypeptides in a histological specimen can be detected by immuno-histochemistry. In this method, the histological specimen is contacted with an anti-CRISP-3 antibody under conditions that allow for formation of CRISP-3 polypeptide-CRISP-3 antibody complex. The CRISP-3 polypeptide-CRISP-3 antibody complex can be detected in various ways. The anti-CRISP-3 antibody can be labeled with a detectable molecule such as a gold particle. Alternatively, the CRISP-3 polypeptide-CRISP-3 antibody complex can be detected using an antibody that recognizes the anti-CRISP-3 antibody. The antibody that recognizes the anti-CRISP-3 antibody can be labeled with any appropriate label.

The presence of a CRISP-3 polypeptide in a biological sample also can be determined by antibody-based sandwich assays. In an antibody-based sandwich assay, the antibody for the antigen to be detected typically is immobilized oil a surface. A biological sample of interest is then contacted with the immobilized antibody. This is performed under conditions that allow for formation of an antigen-antibody complex if the antigen is present in the biological sample. This complex can be detected using a second antibody that recognizes a different epitope on the same antigen. The second antibody can be labeled with any appropriate label. In an ELISA, for example, the second antibody can be labeled with an enzyme having a readily detectable activity. The enzyme typically is alkaline phosphatase or peroxidase. The second antibody also can be labeled with a fluorescent or radioactive molecule. Alternatively, the second antibody can be unlabeled and detected using a third antibody that is labeled and that recognizes the second antibody.

Antibody-based assays for detecting a CRISP-3 polypeptide in a biological sample can be performed qualitatively, in which only the presence or absence of the polypeptide in a biological sample is determined. Alternatively, antibody-based assays also can be performed quantitatively to determine the level of CRISP-3 polypeptide in a sample.

Antibody-based assays can be performed quantitatively by the addition of a labeled competing substrate in the assay. In these antibody-based competitive type assays, typically, the competing substrate is a known amount of the polypeptide the level of which is being measured, and is differentiated from the polypeptide by a label. The labeled competing substrate competes with the unlabeled polypeptide for binding to the antibody. Typically, the level of the labeled competing substrate is known while the level of the polypeptide is reflected in the magnitude of the decrease in detectable levels of competing substrate-antibody complexes. Since the competing substrate competes with the polypeptide for binding to a specific antibody, the more competing substrate-antibody complexes detected, the lower the level of the polypeptide in a sample.

Alternatively, a standard substrate can be used with an antibody-based assay to quantitate the level of a polypeptide. Typically, multiple assays are performed using various known levels of a standard substrate. Known levels of the standard substrate can be correlated with the corresponding results of the assays and this correlation can be used to determine the actual level of a polypeptide in a sample. The standard substrate can be known quantities of the polypeptide or known quantities of a molecule that is recognized by the same antibody as the polypeptide.

Immunoassay methods such as ELISA, radioimmunoassays and Western blotting are described in Chapter 11 of Short Protocols in Molecular Biology (1992) Ausubel, F. M. et al. (eds.) Green Publishing Associates and John Wiley & Sons.

4. CRISP-3 Nucleic Acids

The invention also provides nucleic acid molecules that can be used to examine CRISP-3 RNA levels in biological samples. Nucleic acid molecules can be unlabeled or labeled with any appropriate molecules. Appropriate molecules include, without limitation, biotin, digoxigenin, Texas-Red, fluorescein isothiocyanate, and radioactive isotopes such as P³², P³³, H³, C¹⁴, and S³⁵.

One aspect of the invention pertains to isolated nucleic acid molecules that can be used as primers for synthesis of CRISP-3 nucleic acids. “CRISP-3 nucleic acids” include DNA and RNA molecules that have the CRISP-3 polypeptide-coding region or a portion of the CRISP-3 polypeptide-coding region. CRISP-3 nucleic acids can include sequences corresponding to untranslated regions, translated regions, or both translated and untranslated regions of the CRISP-3 mRNA molecule. CRISP-3 nucleic acids can be single stranded or double stranded.

A primer is a nucleic acid molecule that is complementary to a second nucleic acid molecule and can act as a point of initiation for synthesis of a DNA or RNA form of that second nucleic acid molecule. In this context, the second nucleic acid molecule is referred to as a nucleic acid template. A primer must be sufficiently long to support synthesis from the nucleic acid template. Typically, primers are ten to more than one hundred bases, preferably twelve to fifty bases, and more preferably fifteen to forty bases. A nucleic acid primer can be a double stranded or a single stranded molecule. If double stranded, the primer can first be denatured, i.e., separate into single strands, prior to initiation of DNA or RNA synthesis. A preferred method for denaturation of double stranded nucleic acids is heating. Typically, any conventional automated DNA or RNA synthesizer can be used to synthesize a nucleic acid primer.

Primers can be used as initiation points for synthesis of DNA or RNA from a nucleic acid template. Synthesis of DNA from an RNA template is referred to as reverse transcription (RT). An RT primer, a primer used in an RT reaction, can be complementary to any region of the RNA template. A useful RT primer, for example, is a poly-T primer that is complementary to the 3′ poly-A tail of the RNA template. An RT primer also can be complementary to a region internal to the template molecule.

Primers also can be used as initiation points for synthesis of nucleic acid molecules in a polymerase chain reaction (PCR). PCR amplification refers to a method in which many copies of a nucleic acid target is made. In PCR, a sense and an antisense primer (i.e., a primer pair) are used as initiation points of nucleic acid synthesis. General PCR methods are described in pages 14.2-14.21 of Sambrook et al. (1989) Molecular Cloning, 2nd edition, Cold Spring Harbor Laboratory.

Typically, sequence information from the ends of the region to be amplified or beyond the region to be amplified is used in primer design. Each primer of the pair is designed to be complementary to relative positions along the nucleic acid template such that the nucleic acid product synthesized from one primer can serve as a template for synthesis from the second primer of the pair. Each primer must be sufficiently complementary to the nucleic acid template that contains the segment to be amplified so that, under the appropriate synthesis reaction conditions, the primer will hybridize with the nucleic acid template. A primer can have a 5′ terminus that does not hybridize to the nucleic acid template as long as the 3′ terminus is sufficiently complementary to the template that it will hybridize and allow initiation of nucleic acid synthesis under conditions of the reaction. A primer can have one or more bases that are not complementary to the corresponding bases in the target nucleic acid provided there is sufficient complementarity for hybridization and initiation of nucleic acid synthesis. The non-complementarity bases can be interspersed throughout the primer sequence. Primers that are completely complementary to a portion of the nucleic acid template are preferred. Each primer in a PCR primer pair is designed in a way that the 3′ end of one primer is not complementary to the 3′ end of the other primer to avoid formation of primer-primer-duplexes.

Nucleic acid primers that can act as initiation points for synthesis of CRISP-3 nucleic acids are referred to as CRISP-3 primers. CRISP-3 primers can be any length described above. CRISP-3 primers will hybridize with a CRISP-3 nucleic acid template and act as initiation points for CRISP-3 nucleic acid synthesis under the appropriate conditions. Under the same conditions, CRISP-3 primers will not hybridize to a nucleic acid template that encodes another member of the CRISP family, and cannot function as initiation points for nucleic acid synthesis of this other member of the CRISP family. For example, CRISP-3 primers will not hybridize to a CRISP-1 or CRISP-2 nucleic acid template, and cannot function as initiation points for CRISP-1 or CRISP-2 nucleic acid synthesis.

A CRISP-3 nucleic acid, generated by an RT reaction or RT-PCR amplification, can have the sequence of the entire CRISP-3 RNA molecule. The CRISP-3 nucleic acid also can have the sequence of a portion of the translated, the untranslated, or a portion of both translated and untranslated regions of a CRISP-3 RNA molecule. The CRISP-3 nucleic acid also can have a sequence that is complementary to the entire CRISP-3 RNA molecule or any region of the CRISP-3 RNA molecule.

The invention also encompasses nucleic acids that can be used as hybridization probes to identify CRISP-3 nucleic acids. Hybridization probes that are useful for identifying CRISP-3 nucleic acids are referred to as CRISP-3 hybridization probes. CRISP-3 hybridization probes can be DNA or RNA (e.g. a ribo-probe), and can be double stranded or single stranded molecules. If double stranded, the CRISP-3 hybridization probe can first be denatured, i.e., separate into single strands, prior to use. A preferred method for denaturation of double stranded nucleic acids is heating. A useful CRISP-3 hybridization probe is one that hybridizes to a CRISP-3 nucleic acid under high or moderate stringency conditions, but will not hybridize to a nucleic acid corresponding to another member of the CRISP family, for example, a CRISP-1 or CRISP-2 nucleic acid, under the same stringency conditions. Preferably, the CRISP-3 hybridization probe hybridizes to the 3′ untranslated region of the CRISP-3 RNA but not to the 3′ untranslated region of CRISP-1 or CRISP-2 RNA under high or moderate stringency conditions.

Under high stringency conditions only nucleic acids that have a high degree of homology to the probe will hybridize to the probe. High stringency conditions can include the use of low ionic strength and high temperature for washing, for example, 0.015 M NaCl/0.0015 M sodium citrate (0.1×SSC); 0.1% sodium lauryl sulfate (SDS) at 50-65° C.

Under moderate stringency conditions, nucleic acids that have a lower degree of identity to the probe also will hybridize. Moderate stringency conditions can include the use of higher ionic strength and/or lower temperatures for washing of the hybridization membrane, compared to the ionic strength and temperatures used for high stringency hybridization. For example, a moderate stringency wash procedure consists of 2×SSC at 45° C. for 10 minutes, 1×SSC at 45° C. for 5 minutes, and 0.5×SSC for 45° C. for 5 minutes.

Useful probes typically hybridize to regions of a nucleic acid of interest that have low homology with other nucleic acids. Regions of the CRISP-3 RNA that have low homology with other nucleic acids, and therefore can be used to design CRISP-3 hybridization probes, can be identified by BLAST analysis against HTGS and NR databases. For example, the CRISP-3 cDNA sequence can be subjected to BLASTN analysis against a NR database (http://www.ncbi.nlm.nih.gov/BLAST/). Default values can be used. In this way, a region defined by nucleotides 650 to 1450 of SEQ ID NO: 1 of the CRISP-3 cDNA (GenBank Accession No. NM_{—}006061) is found to have low or no significant homology to other nucleic acid molecules and, therefore, can be useful for designing probes.

The CRISP-3 hybridization probe can be any length useful for specific hybridization under high or moderate stringency conditions. Probes can range from 15 to 400 nucleotides in length and can be, for example, 15, 20, 30, 40, 50, 75, 1 00, 150, 200, 300, or 400 nucleotides in length. Probes greater than 400 nucleotides in length also are useful.

The CRISP-3 hybridization probe can be labeled with any appropriate nucleic acid labeling molecule, for example, a radioisotope, a colorimetric reagent, a fluorescent reagent, or an immunogenic hapten. A labeled CRISP-3 hybridization probe can be detected by standard methods, for example, autoradiography, color development reactions, chemiluminescent reactions, or antibody-based assays. An example of a useful CRISP-3 hybridization probe is a digoxigenin labeled RNA molecule that is antisense to the CRISP-3 RNA.

A CRISP-3 hybridization probe can be obtained by any method. For example, the hybridization probe can be synthesized using an automated nucleic acid synthesizer. The probe also can be generated by PCR amplification from a CRISP-3 nucleic acid. The hybridization probe can be obtained from restriction digestion of genomic DNA, cellular RNA, or a recombinant nucleic acid molecule. The probe also can be amplified from cDNA, cellular RNA, or genomic DNA using appropriate primers and standard PCR methods. DNA and RNA can be extracted from a cell sample using routine methods including, for example, phenol extraction. Genomic DNA and RNA also can be isolated using any commercially available DNA or RNA isolation kit.

5. Nucleic Acid-based Assays

CRISP-3 nucleic acids can be used with any method known in the art to examine CRISP-3 RNA expression. For example, CRISP-3 RNA can be examined by in situ RNA hybridization or by conventional northern hybridization. In both methods, an RNA sample is contacted with a labeled nucleic acid hybridization probe under conditions that allow for complex formation if an RNA target complementary to the labeled nucleic acid hybridization probe is present in the sample. The complex that is formed consists of the nucleic acid hybridization probe and the RNA target.

For in situ hybridization, cells in a histological specimen that express CRISP-3 RNA can be identified using nucleic acids of the invention. A histological specimen can be fixed and embedded, or frozen, and can contain cells that express CRISP-3 RNA as well as cells that do not express CRISP-3 RNA. When a histological specimen is contacted with a labeled CRISP-3 hybridization probe, a cell expressing CRISP-3 RNA is differentiated from one that does not by the presence of a complex consisting of a labeled CRISP-3 hybridization probe and a CRISP-3 RNA molecule. No label or only background levels of label are observed in a cell that does not express CRISP-3 RNA.

Northern hybridization is another method that can be used for identifying cells expressing CRISP-3 RNA. To perform Northern hybridization, cellular RNA is isolated from a cell sample by any standard method. The isolated RNA is separated on an appropriate gel matrix, for example, acrylamide or agarose. Next, the separated RNA is transferred to a membrane and the membrane is contacted with a labeled CRISP-3 nucleic acid hybridization probe under the appropriate conditions. If a CRISP-3 RNA is present, a complex consisting of CRISP-3 RNA and CRISP-3 hybridization probe is formed.

The presence of CRISP-3 RNA also can be detected by any nucleic amplification method known in the art, for example, RT-PCR, TAS (Kwoh et al. (1989) Proc Natl Acad Sci USA 86:1173-7), 3SR (Curr Opin Biotechnol (1993) 4:41-7), SDA (PCR Methods Appl (1993) 3:1-6), LCR (Ann Biol Clin (Paris) (1993) 51:821-6), RCR (U.S. Pat. No. 6,004,826), and BDA (U.S. Pat. No. 5,470,724).

The presence of CRISP-3 RNA also can be determined by any nucleic acid quantitation method known in the art. Methods for quantitating nucleic acids include, without limitation, the mRNA Invader Assay (Third Wave Technologies, Inc.) and various quantitative PCR-based techniques. An example of a quantitative PCR-based technique is the AmpliSensor Assay described in pages 193-202 of PCR Primer: A Laboratory Manual (1995) Dieffenbach, C. and Dveksler, G. (eds.) Cold Spring Harbor Laboratory Press. See also pages 14.30-14.35 of Sambrook et al., (1989) Molecular Cloning, 2nd edition, Cold Spring Harbor Laboratory.

Another quantitative PCR-based technique is real time PCR. In real time PCR, the level of a nucleic acid template in a sample is reflected in the cycle number (C_{T}) at which a threshold level of amplification product is obtained. In general, the higher the level of the nucleic acid template in a sample, the lower the C_{T} value for the amplification reaction. Relative levels of a nucleic acid template in different samples can be determined by comparing the C_{T} for the different samples. Real time PCR can be performed using various commercially available reagents and instruments such as the Taq Man Universal PCR Master Mix (Roche), Taq Man probes (IDT), and the 7700 system. Other real time PCR systems include, for example, the LightCycler (Roche Molecular Biochemicals) and iCycler (Bio-rad).

Another quantitative PCR method involves the use of a control template that is co-amplified in the same reaction as the RNA template of interest. For example, a known amount of a control nucleic acid template can be included with the RNA template of interest in the RT-PCR amplification reaction. The control template is reverse transcribed and co-amplified with the template of interest. Although the control template has substantially the same nucleic acid sequence and primer-binding site as the template of interest and can be amplified using the same primer set, the control template has features that can be used to distinguish it from the template of interest. Features that can be used to distinguish one nucleic acid sample from another include, without limitation, size, and the presence or absence of restriction recognition sequences. The control template can be derived from the template of interest by removal of a nucleic acid segment in the template of interest that is internal to the primer binding sites such that the control template and the template of interest can be differentiated by size. The control template also can be derived from the template of interest by the addition of a nucleic acid segment to a site located between the primer binding sites on the template of interest. The control nucleic acid also can be derived from the template of interest by addition or removal of a restriction enzyme recognition site in the template of interest so that the control and nucleic acid of interest can be differentiated by restriction enzyme digestion.

When a control template is used, a series of RT-PCR amplifications involving co-amplification of known amounts of the control template with unknown amounts of a template of interest can be performed. Typically, various known amounts of the control template are used. The amounts of RT-PCR products obtained for particular amounts of starting control template, i.e., the relative amounts of control nucleic acids, are indicative of the efficiency of the RT-PCR reaction. The relative amounts of control nucleic acids can be used to determine the original amount of the template of interest based on the amount of RT-PCR product obtained by RT-PCR from the template of interest.

6. Polypeptide Profiling

In addition to antibody-based and nucleic acid-based assays, CRISP-3 polypeptides also can be detected using mass spectrometry. Mass spectrometry can be used to detect the presence or absence of CRISP-3 polypeptides in a biological sample. Mass spectrometry also can be used to obtain a profile reflecting polypeptide levels that are indicative of a cancerous condition. For example, a biological sample from a mammal having cancer can be analyzed using mass spectrometry. The resulting mass spectrum is compared with the mass spectrum of a sample obtained from a healthy mammal, and a polypeptide profile representative of a cancerous condition is obtained. The polypeptide profile representative of a cancerous condition consists of polypeptide levels, e.g. CRISP-3 levels, that are observed in the spectrum of the sample from the mammal having cancer, but that differ from those levels observed in the spectrum of the sample from the healthy mammal.

7. Clinical Applications

The invention provides methods and materials for determining cancer diagnosis and prognosis based on examining the levels of CRISP-3 RNA and polypeptides. CRISP-3 polypeptides can be detected in body fluids such as blood, bone marrow, urine, semen, vaginal secretion, saliva, pancreatic fluid, cerebrospinal fluid, or any organ fluid or secretion. CRISP-3 polypeptides can be detected using antibody-based assays discussed above. Typically, levels of CRISP-3 polypeptides in particular body fluids are used to determine a cancer diagnosis or prognosis. For example, detecting elevated levels of CRISP-3 polypeptides in semen can indicate prostate cancer, while quantitating the level of elevation can provide an indication of the progression stage of the cancel.

CRISP-3 RNA can be detected in biological samples that contain cells. CRISP-3 RNA in a histological sample typically is detected using in situ hybridization. CRISP-3 RNA in a biopsy sample or in cells isolated from a body fluid can be detected by isolating cellular RNA and performing nucleic acid-based assays such as Northern analysis or PCR-based assays discussed in the above sections. Cell samples can be obtained from any tissue, for example, blood, prostate, ovary, pancreas, salivary gland, lung, thymus, and descending colon.

In determining cancer diagnosis or prognosis, levels of CRISP-3 RNA or polypeptides can be compared with reference values representing levels of CRISP-3 RNA or polypeptides typically observed in cancer free samples. Levels of CRISP-3 RNA or polypeptides also can be compared with levels typically observed in various stages of cancer. A reference value also can be a level of CRISP-3 RNA or polypeptide determined for an earlier sample from the same patient.

Levels of CRISP-3 RNA or polypeptides can be used with other data to determine a cancer diagnosis or prognosis. For example, levels of CRISP-3 RNA or polypeptides can be used in combination with levels of PSA RNA or polypeptides. Typically, low levels of CRISP-3 and PSA indicate a good prognosis while high levels of CRISP-3 and PSA indicate prostate cancer. In addition, low levels of CRISP-3 with high levels of PSA can indicate BPN while high levels of CRISP-3 and low levels of PSA can indicate pancreas cancer.

Having implicated CRISP-3 in cancel, CRISP-3 genomic DNA, cDNA, RNA, or ESTs can be examined for the presence of nucleotide sequence variants or variant profiles associated with cancer. Nucleotide sequence variants are alterations in the coding and non-coding regions such as exons, introns, and untranslated sequences when compared to a wild type sequence. Nucleotide sequence variants can be substitutions, deletions, or insertions of one or more nucleotides. A single nucleotide variant is a deletion, an insertion, or a substitution at a single base pair site, i.e., a single nucleotide polymorphism (SNP). A “variant profile” refers to a pattern of nucleotide sequence variants observed in a particular gene or other nucleotide sequence of interest.

CRISP-3 nucleotide sequence variants can be identified in various ways, for example, by comparing CRISP-3 ESTs generated from different libraries or by comparing CRISP-3 genomic DNA sequences of different individuals. CRISP-3 EST sequences can be obtained from a human EST database while genomic DNA sequences can be obtained from the human genome project. Once a CRISP-3 nucleotide sequence variant or variant profile is identified, the CRISP-3 nucleotide sequence variant or variant profile can be assessed to determine whether there is a correlation between the occurrence of this particular variant or variant profile and the occurrence of cancer. This can be done by examining the source of the DNA from which the sequence variant or variant profile is obtained. For example, the finding of a statistically significant frequency of occurrence of a particular CRISP-3 sequence variant or variant profile in cancer libraries but not in normal libraries indicates a correlation with a cancer condition. In this case, the particular CRISP-3 sequence variant or variant profile can be used to identify patients who are predisposed to cancer.

To determine if a patient has a particular CRISP-3 sequence variant or a variant profile associated with cancer, genomic DNA or RNA isolated from the patient can be analyzed for the presence of nucleotide variants using any known method. Typically, an amplification step is performed before proceeding with the detection method. Detection methods include, for example, sequencing exons, introns, 5′ untranslated sequences, or 3′ untranslated sequences as well as performing allele-specific hybridization, allele-specific restriction digests, and mutation-specific polymerase chain reactions (MSPCR). An example of a useful sequencing method for detecting sequence variants is the dye primer sequencing method typically used for increased accuracy of detecting heterozygous samples. The method of allele-specific hybridization is described in Stoneking et al. (1991) Am. J. Hum. Genet. 48:370-382 and Prince et al. (2001) Genome Res 11:152-162. For nucleotide sequence variants that introduce, remove, or alter a restriction recognition site, restriction digest with an appropriate restriction enzyme can differentiate between different alleles. For nucleotide sequence variants that do not introduce, remove, or alter a common restriction site, mutagenic amplification primers can be designed that will introduce, remove, or alter a restriction recognition site when the variant allele is amplified or when the wild type allele is amplified. Once amplified, the variant allele and the wild type allele can be distinguished by digestion with the appropriate restriction endonuclease.

Nucleotide sequence variants arising from insertions or deletions of one or more nucleotides can be identified by examining the size of the DNA fragment encompassing the insertion or deletion. Typically, the region encompassing the insertion or deletion is amplified and then the size of the amplified product is determined by electrophoretic separation and comparison with size standards. To facilitate visualization, one of the primers can be labeled with, for example, a fluorescent moiety. Size standards can be labeled with a fluorescent moiety that differs from that of the primer.

In allele-specific PCR or MSPCR, specific primers and PCR conditions can be designed to amplify a product only when the variant allele is present or only when the wild type allele is present. For example, DNA from a patient sample and a control sample can be amplified separately using either a wild type primer or a primer specific for the variant allele. Each set of reactions is then examined for the presence of amplification products using standard DNA visualization methods. For example, the amplification products can be separated by electrophoresis and visualized by staining with ethidium bromide or other DNA intercalating dye. In DNA samples from heterozygous patients, reaction products would be detected in the reaction in which a wild type primer was used as well as in the reaction in which a primer specific for the variant allele was used. Patient samples containing solely the wild type allele would have amplification products only in the reaction in which the wild type primer was used. Similarly, patient samples containing solely the variant allele would have amplification products only in the reaction in which the variant primer was used.

In addition, CRISP-3 nucleotide sequence variants can be detected by single-stranded conformational polymorphism (SSCP) detection (Schafer et al. (1995) Nat. Biotechnol. 15:33-39), denaturing high performance liquid chromatography (DHPLC, Underhill et al. (1997) Genome Res. 7:996-1005), infrared matrix-assisted laser desorption/ionization (IR-MALDI) mass spectrometry (WO 99/57318), and combinations of such methods. CRISP-3 nucleotide sequence variants also can be detected indirectly using antibodies that have specific binding affinity for variant CRISP-3 polypeptides.

The invention also provides kits that contain the necessary reagents for detection of CRISP-3 RNA or polypeptides in a biological sample. Reagents can include anti-CRISP-3 antibodies and CRISP-3 nucleic acids such as primers and hybridization probes. In addition, kits can contain anti-PSA antibodies and PSA nucleic acids useful for identifying PSA RNA. Kits provided by the invention also can contain a reference value or a set of reference values indicating normal and various clinical progression stages of cancer. Kits can have positive controls, negative controls, or both positive and negative controls for comparison with the test sample. A negative control can be a sample that does not have a CRISP-3 RNA or polypeptide. A positive control can be a sample or samples containing various known levels of CRISP-3 RNA or polypeptides. Kits can contain any combinations of CRISP-3 antibodies, CRISP-3 nucleic acids, PSA antibodies, and PSA nucleic acids.

The invention will be further described in the following examples, which do not limit the scope of the invention described in the claims.

### EXAMPLES

### Example 1

Computational Analysis of EST Libraries Generated from Normal and Cancerous Prostate Tissues Shows that CRISP-3 EST is Predominately Found in Prostate Cancer Tissue Libraries

To identify genes that are expressed differentially in normal and cancerous tissues, EST databases, generated from the RNA of more than 50 different normal and cancerous human tissues and organs, were analyzed. A computational software was developed to sort each EST in the human EST database into clusters, each cluster consisting of ESTs corresponding to a single gene. The tissue distribution of each EST within a cluster was then determined. The sequence of an EST also was analyzed by BLASTN analysis (http://www.ncbi.nlm.nih.gov/BLAST/) to determine whether the EST corresponds to a known gene.

About 70 000 ESTs have been identified from normal prostate cells, prostate cancer cells, and prostate cell-lines, and the corresponding sequences are available in various public databases. The analysis was confined to micro-dissected prostate libraries to avoid erroneous calculation of expression levels due to contamination of non-epithielial cells. Hundreds of ESTs, including those that correspond to novel genes not known to associate with prostate cancer, were identified. ESTs showing tissue specificity and corresponding to genes that encode secretory polypeptides were selected.

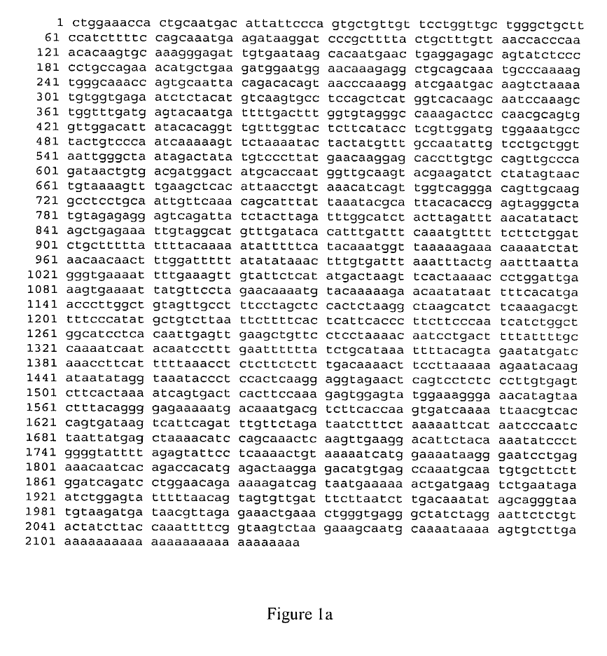

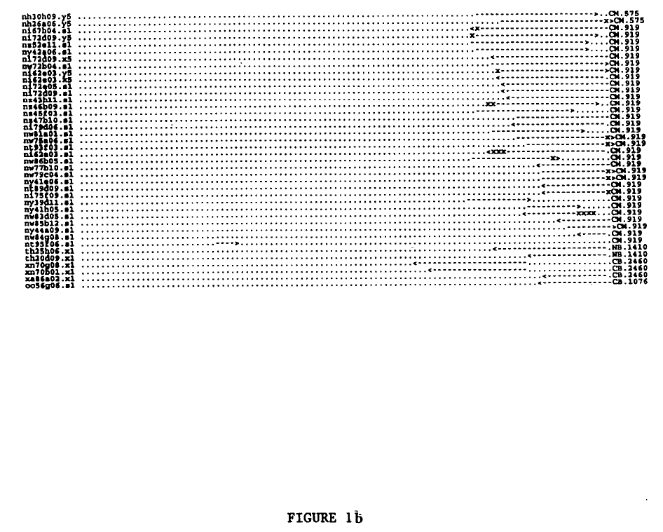

Forty ESTs corresponding to the CRISP-3 gene (GenBank accession no. NM_{—}006061) were found in the NCBI human EST database. The regions of overlap between the forty EST sequences and the full-length CRISP-3 cDNA sequence are illustrated in FIG. 1. Of these, thirty-six were derived from prostate cells. Of the thirty-six, thirty-four were derived from cancerous prostate tissues while two were derived from normal bulk tissues. Of the thirty-four CRISP-3 ESTs derived from cancerous prostate tissues, two were from microdissected malignant prostate cells while the remaining thirty-two were derived from a needle biopsy of a metastatic lesion of the bone. The metastatic lesion of the bone originated from a prostate carcinoma. The finding that the majority of CRISP-3 ESTs in the database was derived from cancerous prostate tissues shows that CRISP-3 polypeptide is expressed at a higher level in cancerous prostate cells than in normal prostate cells. In addition to prostate tissues, CRISP-3 ESTs also were found in blood CML (chronic myelogenous leukemia) and lung (cancer) libraries.

### Example 2

Computational Analysis of CRISP-3, PSA, HK2, and GAPDH ESTs Shows that the CRISP-3 mRNA is Transcribed at a Higher Level in Cancerous Prostate Cells than in Normal Prostate Cells.

The level of expression of an mRNA in a tissue can be estimated by determining the number of ESTs corresponding to the mRNA in an EST library generated from the tissue. The difference in the number of ESTs corresponding to a particular mRNA between an EST library generated from normal tissue and an EST library generated from diseased tissue indicates changes in the level of mRNA expression due to disease.

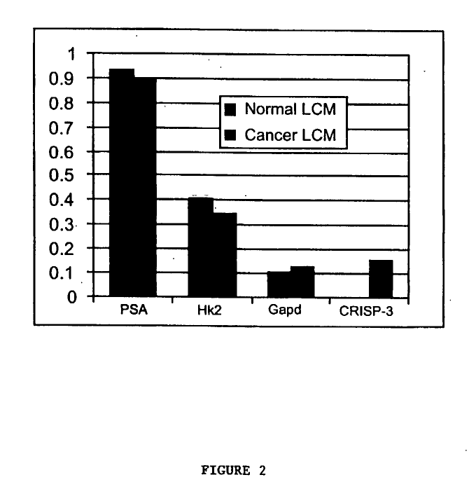

Using this method, the expression levels of CRISP-3, PSA, HK2 (human kallikrein, and glyceraldehyde phosphate dehydrogenase (GAPDH) were estimated by electronic profiling of data from EST libraries. EST libraries used in the analysis were generated from normal and cancerous prostate cells obtained by microdissection. Electronic profiling results indicate that expression levels of PSA, HK2, and GAPDH per cell were comparable between normal and cancerous prostate cells. The levels of PSA mRNA in normal and cancer samples were comparable and accounted for about 1% of the total cellular mRNA. HK2 mRNA levels in normal and cancer samples also were similar and accounted for about 0.4% of total cellular mRNA. Similarly, GAPDH mRNA levels in normal and cancer samples were similar and account for about 0.1% of cellular mRNA. In contrast, the levels of CRISP-3 mRNA in all cancer samples were significantly greater than in normal samples. CRISP-3 mRNA levels in normal samples could not be calculated since no CRISP-3 EST was found in normal microdissected libraries. Since a small number of CRISP-3 ESTs was found in normal bulk libraries, it is likely that CRISP-3 mRNA is expressed at a low level in normal epithelial cells. The elevated level of CRISP-3 mRNA expression in cancerous samples shows that CRISP-3 is a stronger marker for prostate cancer than PSA or HK2. (FIG. 2)

### Example 3

RT-PCR Experiments Show that Cancerous Prostate Epithelial Cells Transcribed More CRISP-3 mRNA than Normal Prostate Epithelial Cells

To verify bioinformatic results showing that cancerous prostate cells expressed a higher level of CRISP-3 mRNA than normal prostate cells, RT-PCR was performed. CRISP-3 mRNA expression by pure populations of normal and cancerous prostate epithelial cells was measured by RT-PCR. Pure populations of prostate epithelial cells used in RT-PCR experiments were obtained using Laser Capture Microdissection (LCM) (Simon et al. (1998) Trends in Genetics 14:272 and Emmert-Buck et al. (1996) Science 274:998-1001). The LCM technique allowed for isolating normal and cancerous prostate epithelial cells precisely and efficiently from among a combination of normal, diseased, epithelial and non-epithelial cells.

Normal and cancerous prostate epithelial cell samples from five different prostate cancer cases were examined. The cancerous prostate epithelial cells had a Gleason Score of 6 (Biopsy Pathology of the Prostate (1997) Bostwick, D. A. (ed.) Chapman and Hall. For each case, total RNA was isolated from two thousand LCM captured normal and two thousand LCM captured cancerous prostate epithelial cells. Total RNA from normal or cancerous prostate epithelial cells was used as the starting substrate in the RT-PCR experiment. Oligo-dT primers were used to reverse transcribe GAPDH, PSA, and CRISP-3 mRNAs into their corresponding cDNA products. The same oligo-dT primer, consisting of 12 to 17 dTTP residues, was used for reverse transcription of GAPDH, PSA, and CRISP-3 mRNA. In the reverse transcription (RT) reaction, GAPDH, PSA, and CRISP-3 mRNAs were reverse transcribed using 200 units of Super-Script II (Life Technologies), 25 ng/μL of oligo-dT₁₅₋₁₈, and 0.5 mM of each dNTP in a 20 μL final reaction volume. RT reactions were performed at 42° C. for 60 minutes.

Following the RT reaction, forty rounds of PCR amplification were performed using gene specific primers. Sequences of the forward and reverse primers, respectively, were as follows.

| | | |
|---|---|---|
| GAPDH: | 5′-CGAGATCCCTCCAAAATCAA-3′ | (SEQ ID NO:2) and |
| | 5′-ATCCACAGTCTTCTGGGTGG-3′ | (SEQ ID NO:3) |
| PSA: | 5′-ATTGTGGGAGGCTGGGAGTG-3′ | (SEQ ID NO:4) and |
| | 5′-GTCACCTTCTGAGGGTGAAC-3′ | (SEQ ID NO:5) |
| CRISP-3: | 5′-ATGTGAGCCAAATGCAATGT-3′ | (SEQ ID NO:6) and |
| | 5′-CATTACCCTGCTATATTTGTCAAGA-3′ | (SEQ ID NO:7) |

PCR amplifications were performed with 1 μL of the RT reaction, 1 unit of Taq Polymerase (Roche), 0.5 μM of each primer, 0.5 mM of each dNTP and 1 μL PCR buffer. PCR amplification included an initial denaturation step, 40 cycles of amplification, and a final extension step. The denaturation step was 1 minute at 94° C. Each amplification cycle consisted of 30 seconds at 94° C., 30 seconds at 59° C., and 40 seconds at 72° C. The final extension step was 7 minutes at 72° C. Amplified products corresponded to nucleotides 298 to 627 of GAPDH (GenBank Accession No. M33197), 378 to 883 of PSA (GenBank Accession No. X07730), and 1834 to 1982 of CRISP-3 (GenBank Accession No. NM_{—}006061).

The amounts of GAPDH, PSA and CRISP-3 cDNA products obtained from RT-PCR were analyzed by gel electrophoresis. An equal amount of GAPDH cDNA product was obtained in normal and cancerous prostate epithelial cell samples indicating that comparable levels of GAPDH mRNA were expressed. Similarly, an equal amount of PSA cDNA product was obtained in normal and cancerous prostate epithelial cell samples, also indicating that comparable levels of PSA mRNA were expressed. Since only prostate epithelial cells produce PSA, the similar level of PSA mRNA expression in normal and cancerous samples confirmed that pure populations of epithelial cells were used in the experiment. In contrast to GAPDH and PSA, the amount of CRISP-3 cDNA product amplified from total RNA of cancerous prostate epithelial cells was significantly greater than the amount of CRISP-3 cDNA product amplified from total RNA of normal prostate epithelial cells. In most cases, the CRISP-3 cDNA product amplified from total RNA of normal prostate epithelial cells was barely visible when analyzed by gel electrophoresis. A signal was detected, however, in all cases in which total RNA from cancerous prostate epithelial cells was used. This result indicates that cancerous prostate epithelial cells express detectably more CRISP-3 mRNA than normal prostate epithelial cells.

### Example 4

Real Time RT-PCR Experiments Indicate at Least Eight Fold Elevation of CRISP-3 Polypeptide Expression Level in Cancerous Prostrate Epithelial Cells

To obtain a more accurate estimate of the change in CRISP-3 mRNA expression level in cancerous prostate epithelial cells with respect to normal/benign prostate epithelial cells, real time RT-PCR was used to compare relative expression levels of CRISP-3 mRNA in the two different cell types.

In real time RT-PCR, the number of PCR cycles needed to generate a threshold level (C_{T}) of an amplification product is an indication of the expression level of the mRNA that is amplified. A relative expression level of an mRNA in two different samples is estimated by comparing the C_{T} values. The difference in C_{T} values of two samples is represented by Δ(C_{T}).

To examine the expression level of CRISP-3 mRNA, an 89 base pair region corresponding to nucleotides 1773 to 1862 of the 3′ untranslated region of the CRISP-3 mRNA was selected for real time RT-PCR amplification. The 3′ untranslated region is specific to the CRISP-3 mRNA and does not show homology with mRNAs encoding CRISP-1 and CRISP-2. As controls, expression levels of PSA and GAPDH mRNA in cancerous and normal/benign prostate epithelial cells also were analyzed by real time RT-PCR amplification. Fragments corresponding to nucleotides 1561 to 1670 of PSA and 549 to 656 of GAPDH were generated in the reaction.

Total mRNA used in the amplification experiment was isolated as described in Example 3. Approximately equal numbers of epithelial cells were micro-dissected from cancerous and normal/benign prostate samples. Six different cases, five having Gleason Scores of 6 and one having a Gleason Score 9, were evaluated. Real time RT-PCR was performed using the TaqMan Universal PCR Master Mix (PE Biosystems) and the TaqMan PE 7700 system. Primers used for amplification of the 3′ un-translated tail of the CRISP-3 mRNA and segments of PSA and GAPDH mRNA were designed using the Primer Express software (PE Biosystems). Sequences of the forward and reverse primers, respectively, were as follows.

| | | |
|---|---|---|
| GAPDH: | 5′-CATCCATGACAACTTTGGTATCGT-3′ | (SEQ ID NO:8) and |
| | 5′-CCATCACGCCACAGTTTCC-3′ | (SEQ ID NO:9) |
| PSA: | 5′-GGTTGTCTGGAGGACTTCAATACA-3′ | (SEQ ID NO:10) and |
| | 5′-GAGGGAGGGTCTTCCTTTGG-3′ | (SEQ ID NO:11) |
| CRISP-3: | 5′-AAATCATGGAAAATAAGGGAATCCT-3′ | (SEQ ID NO:12) and |
| | 5′-CCAAGAAGCACATTGCATTTG-3′ | (SEQ ID NO:13) |

The dual-labeled TaqMan probes used in monitoring the amplification reactions were obtained from IDT and had the following sequences.

| | | |
|---|---|---|
| GAPDH: | 5′-AAGGACTCATGACCACAGTCCATGCCA-3′ | (SEQ ID NO:14) |
| PSA: | 5′-ACTGACCCCCTGGAAGCTGATTCACTATG-3′ | (SEQ ID NO:15) |
| CRISP-3: | 5′-AGAAACAATCACAGACCACATGAGACTAAGGAGACA-3′ | (SEQ ID NO:16) |

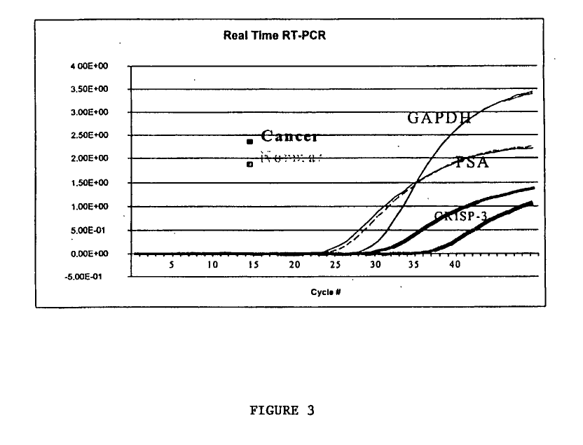

FIG. 3 illustrates results obtained for a representative prostate cancer case having a Gleason Score of 6. The C_{T} value for amplification of a segment of the GAPDH mRNA from cancerous prostate epithelial cells is comparable to the C_{T} value for amplification of the same from normal/benign prostate epithelial cells. As a house keeping gene product, GAPDH is produced at the same level by benign and cancerous prostate epithelial cells. Therefore, similar C_{T} values for GAPDH mRNA amplification from the two samples indicate that equivalent amounts of total RNA from both types of cells were used in the RT-PCR experiment. Similarly, the C_{T} value for amplification of a segment of the PSA mRNA from cancerous epithelial cells also was comparable to the C_{T} value for amplification of the same from normal/benign prostate epithelial cells. The similar C_{T} values for PSA mRNA amplification from the two samples indicate that normal/benign and cancerous prostate epithelial cells produce similar levels of PSA. Furthermore, the similar C_{T} values also indicate that the two micro-dissected cell samples consisted of fairly pure populations of the two types of epithelial cells. In contrast to the GAPDH and PSA data, the C_{T} value for amplification of the 3′ un-translated region of CRISP-3 mRNA from cancerous prostate epithelial cells was significantly different from the C_{T} value for amplification from normal/benign prostate epithelial cells. For amplification from cancerous prostate epithelial cells, the C_{T} value was 31, i.e., 7 amplification cycles earlier than the C_{T} value of 38 for normal/benign prostate epithelial cells. This difference in C_{T} values (Δ(C_{T})) between cancerous and normal/benign samples corresponds to ten to a hundred-fold elevation of CRISP-3 expression in cancerous prostate epithelial cells when compared with normal/benign prostate epithelial cells depending on amplification efficiency. The fold elevation of expression was determined using the formula: fold elevation=2^{(Δ}^{^{C}}_{T}).

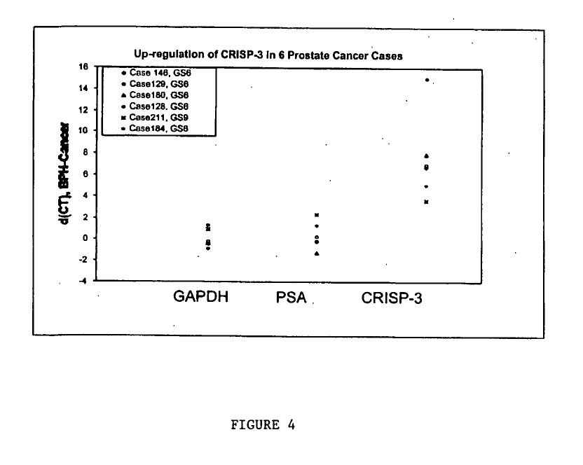

Similar results were obtained in the other five cases shown in FIG. 4. The Δ(C_{T}) values for amplification of PSA and GAPDH mRNAs from total RNA obtained from normal/benign and cancerous cells (normal/BPH—Cancer) ranged from a value of −2 to 2. In contrast, the Δ(C_{T}) values for amplification of the 3, untranslated region of the CRISP-3 mRNA from total RNA obtained from normal/benign and cancerous cells ranged from 5 to 15 for six prostate cancer cases having a Gleason Score of 6. These Δ(C_{T}) values are statistically significant (paired t-test p<0.01) and correspond with elevations in CRISP-3 mRNA expression levels of 32 to 32,768 fold, respectively. In the case with a Gleason Score of 9, the elevation in CRISP-3 mRNA expression by cancel was 8 fold. These data indicate that CRISP-3 is a more sensitive diagnostic marker for prostate cancer than PSA, as PSA expression is not altered in prostate cancer.

### Example 5

Comparison of CRISP-3 and PSA mRNA Levels in Benign and Cancerous Bulks Tissue Samples

To compare PSA and CRISP-3 mRNA levels in benign and cancerous prostate tissues, PSA and CRISP-3 mRNA levels were determined using real time RT-PCR. Fifteen bulk samples of moderately differentiated adenocarcinoma (Gleason score 6) and matched benign tissues were analyzed. Levels of PSA and CRISP-3 mRNAs were normalized using the level of GAPDH mRNA according to the formula:

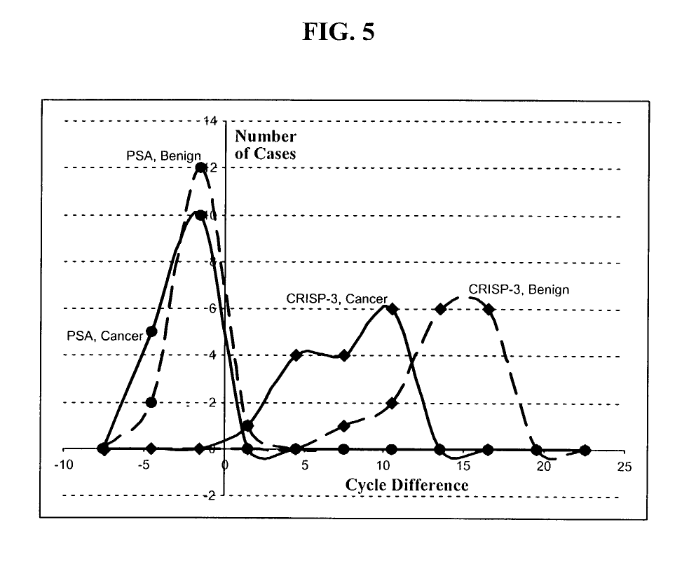

Δ(C_{T})=C_{T-X}−C_{T-GAPDH}

where C_{T-X} is the C_{T} value for PSA or CRISP-3, and Δ(C_{T}), the cycle difference, represents the level of PSA or CRISP-3 mRNA after normalization. Results are shown in FIG. 5. The x-axis indicates the cycle difference, while the y-axis indicates the number of benign or cancerous cases at each cycle difference.

The overlapping curves that represent levels of PSA mRNA in benign and in cancerous samples indicate similar levels of PSA mRNA. The mean of the differences determined between benign and cancerous samples was less than one cycle number (i.e., less than two-fold difference). In contrast, the curves representing levels of the CRISP-3 mRNA in benign and cancerous samples were distinct and mostly non-overlapping indicating that different levels of CRISP-3 mRNA were produced in benign and cancerous samples.

On average, the level of PSA mRNA was 10 to 20 fold higher in benign and cancerous samples than the level of GAPDH mRNA. In contrast, levels of the CRISP-3 mRNA in cancerous and benign samples were ˜50 fold (˜6 cycles) and ˜4000 fold (˜12 cycles) lower than the level of GAPDH mRNA in those samples, respectively. Similar results were obtained in samples of bulk prostate tissue of poorly differentiated adenocarcinoma (data not shown). Since the number of epithelial cells in the benign and cancerous bulk samples was similar, it was concluded that the level of CRISP-3 mRNA was significantly elevated in prostate adenocarcinoma with respect to the level of GAPDH mRNA.

Although CRISP-3 is not as highly expressed as PSA in prostate (FIG. 5), the level of CRISP-3 mRNA is elevated in cancerous relative to benign prostate tissues. Therefore, in patients with prostate cancer, CRISP-3 polypeptide levels will increase in the blood and semen due to an increase in the number of CRISP-3 secreting cancer cells. Unlike PSA, the difference in CRISP-3 production level between cancerous and normal tissues renders it a specific and sensitive diagnostic indicator of prostate cancer. In addition, the elevated serum level of CRISP-3 that accompanies the development of prostate cancer will provide information regarding tumor volume, stage and outcome.

### Example 6

Identification of Cancerous Prostate Epithielial Cells in a Frozen Prostate Tissue Section by in situ Hybridization

Cancerous prostate epithelial cells were identified in a frozen tissue section containing non-epithelial prostate cells, normal prostate epithelial cells and cancerous prostate epithelial cells using in situ hybridization. The probe was a 400 base antisense ribo-probe labeled with digoxigenin-conjugated UTP. The control ribo-probe had a sequence complementary to the antisense ribo-probe and is referred as the sense ribo-probe.

To generate the ribo-probes, the 400 base 3′ UTR of the CRISP-3 RNA was amplified by RT-PCR from RNA isolated from LnCap cell line using two primer sets. The first primer set was designed so that the RT-PCR product had Xho I and Eco RI restriction recognition sites at the 5′ and 3′ end, respectively. The sequences of these primers were as follows:

| | | |
|---|---|---|
| Cr3XE757S: | 5′-GTTGTTCTCGAGCGCATTACACACCGAGTAGG-3′ | (SEQ ID NO:17) |
| Cr3XE1158AS: | 5′-CAACAAGAATTCGCAACTACAGCCAAGGGTTC-3′ | (SEQ ID NO:18) |

The second primer set was designed so that the RT-PCR product had Eco RI and Xho I restriction recognition sites at the 5′ and 3′ end, respectively. The sequences of these primers were as follows:

| | | |
|---|---|---|
| Cr3EX757S: | 5′-CAACAAGAATTCCGCATTACACACCGAGTAGC-3′ | (SEQ ID NO:19) |
| Cr3EX1158AS: | 5′-GTTGTTCTCGAGGCAACTACAGCCAAGGGTTC-3′ | (SEQ ID NO:20) |

The RT-PCR products were cloned into the Eco RI and Xho I sites of the pcDNA3 vector and in vitro transcription of the cloned RT-PCR products were performed using reagents from T7 MAXIscript (Ambion). The ribo-probes generated by in vitro transcription were labeled by inclusion of digoxigenin-UTP in the transcription reaction. The ribo-probes corresponded to a region encompassed by nucleotides 757 to 1158 of the CRISP-3 cDNA sequence (GenBank accession no. NM_{—}006061). This region shows low homology with the corresponding regions in CRISP-1 or 2 cDNA as determined by BLAST analysis using the HTGS and NR databases and also does not include any repeat Alu elements.

The in situ protocol was adopted from Braissant & Wahli ((1998) Biochemica 10:16) with minor modifications. Briefly, frozen prostate tissues in OCT were sectioned at 12 μm on Superfrost/Plus microscope slides (Fisher Part number 12-550-15). Tissue sections were then treated with 4% paraformaldehyde in PBS (phosphate buffered saline) for 20 minutes at room temperature, acetylated for 10 minutes in 0.25% acetic anhydride in 0.1 M triethanolamine (pH 7.9), and washed in PBS for 15 minutes. Sections were equilibrated in 5×SSC and prehybridized at 59° C. for 2.5 hours. Prehybridization buffer contained 50% deionized formamide, 5×SSC, and 100 μg/mL salmon sperm DNA. Hybridization was performed at 59° C. overnight with 250 ng/mL of DIG-labeled riboprobe in prehybridization buffer in a humid chamber containing 50% deionized formamide and 5×SSC. On day 2, slides were incubated for 30 minutes in 2×SSC at room temperature (Gleason score 10 slides were treated with 1 μg/mL RNase A for 10 minutes) and stringently washed with 2×SSC and 0.1×SSC at 64° C. for one hour each. Sections were stained with alkaline phosphatase overnight at room temperature and mounted.

Results show that non-epithelial cells were stained lightly as was the background. Normal epithelial cells were stained darker than the background while the strongest staining was observed with cancerous epithelial cells having a Gleason score of six or greater. The darker staining cancerous prostate epithelial cells confined that CRISP-3 mRNA was expressed at elevated levels in these cells. These observations are in agreement with real time RT-PCR experimental results indicating elevated levels of CRISP-3 expression in cancerous prostate cells.

The expression of CRISP-3 in epithelial cells of prostate adenocarcinoma also was examined using in situ hybridization. Intense cytoplasmic hybridization signals were observed in epithelial cells that corresponded to Gleason scores 6 and 10 adenocarcinoma. Variable weak signals were seen in benign acini including benign prostatic hyperplasia (BPH). Sections that were hybridized with the sense CRISP-3 ribo-probe were not stained. Therefore, CRISP-3 is produced by prostate epithelial adenocarcinoma.

### Example 7

CRISP-3 is Expressed Predominantly in Prostate, Pancreas, and Salivary Gland

Analysis of EST databases showed that CRISP-3 is expressed in normal and cancerous prostate tissues, cancerous lung tissues and blood CML tissues. To determine if CRISP-3 is expressed in other tissues, dot blot analysis was performed using a Multiple Tissue Expression (MTE) Array (ClonTech). The MTE Array contains poly A+ RNA of 76 different human tissues and cancer cell lines immobilized oil a positively charged nylon membrane. To determine if CRISP-3 is expressed in these different tissues, the array was hybridized with a [α-³²P] dCTP labeled probe that recognizes the non-homologous 3′ untranslated region of CRISP-3.

The probe was a PCR product generated by amplification of the 3′ untranslated region of CRISP-3 as described in Example 4. A 25 ng amount of PCR product was labeled with radioactive dCTP. The labeling reaction consisted of 20 μM each of dATP, dTTP, and dGTP; 300 Ci/mmol [α-³²P] dCTP; 0.1 μg/μL of random primers; buffer; and 1 unit of T4 DNA polymerase. The labeling reaction was incubated at 37° C. for 20 minute and then stopped by addition of EDTA.

Prior to hybridization, the MTE array was prehybridized in an ExpressHyb Solution (ClonTech) containing 0.1 mg/mL sheared salmon testes DNA for 30 minute at 65° C. The array was then hybridized with 1×10⁷ cpm of purified probe in pre-hybridization buffer. Hybridization was performed overnight at 65° C.

Results from multiple dot blot hybridization showed that CRISP-3 is expressed predominantly in prostate, pancreas, and salivary gland. CRISP-3 also is expressed in ovary, thymus, fetal thymus, and descending colon gland although in less abundance.

### Example 8

Purification of CRISP-3 Polypeptide

To demonstrate that CRISP-3 is a secretory polypeptide, the expression vector pcDNA3.1GS-CRISP-3-V5-His6 was transiently transfected into HEK293 cells. The expression vector pcDNA3.1GS-CRISP-3-V5-His6 has a gene encoding a 34 kD CRISP-3 polypeptide having a C-terminal V5 tag that can be detected using anti-V5 antibody. Sixty hours after HEK293 cells were transfected with pcDNA3.1GS-CRISP-3-V5-His6, culture supernatant was collected and subjected to western analysis. A vector having no insert (pcDNA3.1GS) was used as negative control. Approximately □g of proteins were used in the western analysis. The presence of CRISP-3-V5-His6 polypeptide in the culture supernatant was detected by anti-V5 antibody.

### Example 9

Generation of a CRISP-3 Polyclonal Antibody by DNA Immunization

Synthetic peptides of CRISP-3, made by the Mayo Clinic Protein Core facility were used to generate polyclonal CRISP-3 antibodies. CRISP-3 synthetic peptide-2 having the amino acid sequence: NYRHSNPKDRMTSLKC (N to C terminus) was conjugated to keyhole limpet hemocyanin (KLH) and then used as an immunogen to generate polyclonal antibody in BALB/c mice. Binding characteristics of the antibody were evaluated by western analysis. A polyclonal antibody, anti-peptide-2 antibody, capable of specifically cross-reacting with the CRISP-3 polypeptide expressed in the LNCap cell line was generated.

Polyclonal antibody to the full-length CRISP-3 polypeptide was generated by DNA immunization using pcDNA3-CRISP-3, a plasmid expressing the full-length CRISP-3 polypeptide. Six Balb/c female mice were immunized using a standard protocol (Chowdhury et al. (2001) J Immunol Methods 249:147-154 and Boyle et al. (1997) Proc Natl Acad Sci USA 94:14626-31). The sera of immunized mice were screened using immunohistochemistry (IHC) for the presence of a polyclonal antibody that would recognize the CRISP-3 polypeptide. The antisera of four mice were found to produce CRISP-3 specific polyclonal antibody in a first level screen. In this first level screen, these antisera were used at a 1:1000 dilution in an immunostaining experiment to identify cancerous prostate epithelial cells from among cancerous and normal prostate epithelial cells as well as non-epithelial cells. The cancerous prostate epithelial cell sample so analyzed had a Gleason Score of 6. Results showed that cancerous epithelial cells were stained with the CRISP-3 specific antisera while non-epithelial cells were not. Furthermore, the specificity of CRISP-3 immunostaining was demonstrated by the absence of staining of the cancerous prostate epithelial cells by a negative control antisera. The negative control antisera were the sera obtained from the same mouse prior to immunization. This CRISP-3 specific polyclonal antibody is used for initial immunohistochemistry studies and CRISP-3 protein purification.

Antiserum containing polyclonal antibody to the CRISP-3 polypeptide also is generated by DNA immunization using the pDISPLAY plasmid (Invitrogen). The CRISP-3 polypeptide that is expressed by the pDISPLAY plasmid is anchored oil the membrane of the expressing cells. This approach enhances the immune reactivity of the polyclonal antibody as the circulating CRISP-3 antibody in the serum is not attached to CRISP-3 polypeptide.

Successful immunostaining of cancerous prostate epithelial cells using a CRISP-3 specific antibody should demonstrate that expression of CRISP-3 polypeptide is elevated in cancerous prostate epithelial cells. Therefore, cancerous prostate epithelial cells can be distinguished from normal prostate epithelial cells by examining the level of CRISP-3 polypeptide as well as the level of CRISP-3 mRNA.

### Example 10

Production of CRISP-3 Specific Monoclonal Antibody

To obtain a monoclonal antibody for CRISP-3, hybridoma technology and phage display techniques are used. Mice immunized with DNA expressing CRISP-3 or with purified CRISP-3 polypeptide that shows strongest polyclonal antibody activity against CRISP-3 are selected for analysis. Splenocytes from selected mice are extracted and used for monoclonal antibody production using both the hybridoma technique and phage display technology; see A Practical Guide to Monoclonal Antibodies (1991) Liddell, J. E. and Cryer, A., John Wiley & Sons.

Hybridoma technology is used to generate a hybridoma clone that produces an antibody specific for CRISP-3. To generate a CRISP-3 producing hybridoma, splenocytes isolated from immunized mice are fused with P3.653 myeloma cells. Those hybridoma clones that produce CRISP-3 specific antibodies are selected using ELISA. CRISP-3 antibody-producing hybridoma clones are selected using ELISA plates coated with purified recombinant CRISP-3. Clones are selected based on strong binding to CRISP-3 and negligible binding with CRISP-2 or CRISP-1. Western blot analysis using denatured and non-denatured SDS-PAGE is used to characterize CRISP-3 specific antibodies.

In addition to hybridoma technology, phage display also is used to generate CRISP-3 specific monoclonal antibody. Splenocytes from mice immunized with DNA or a purified CRISP-3 polypeptide are used for single chain antibody (ScFv) generation Using standard PCR techniques, antibody heavy and light chains are linked together by a 3G₄S linker peptide. Approximately five rounds of panninig will be performed. The selected ScFv fragments are then sequenced and subjected to further in vitro affinity maturation. During affinity maturation, random mutations are generated in the complimentary determining regions (CDR) of the heavy and light chains thereby enhancing the specificity affinity of the antibody. In this way, a CRISP-3 specific ScFv having a K_{d} value of 1-10 nM is generated.

### Example 11

Development of an Immunoassay for CRISP-3

In order for CRISP-3 to be a useful diagnostic marker for prostate cancer, a stringent immunoassay for detecting CRISP-3 in blood, urine, or seminal plasma samples is required. CRISP-3 antibodies are selected using ELISA plates coated with purified recombinant CRISP-3 polypeptides. Clones are selected based on their strong binding to CRISP-3 polypeptide and negligible reactivity with CRISP-2 or CRISP-1 polypeptide. Western blot analysis using denatured and non-denatured SDS-PAGE is used to further screen these antibodies. Clones with strongest immuno-reactivity against native (properly folded) CRISP-3 will be selected for further analysis.

Antibodies that react with different CRISP-3 epitopes are tested in a pair wise manner to develop highly sensitive and specific sandwich immuno-assays. For sandwich immuno-assays, antibodies can be immobilized on solid phase materials such as paramagnetic particles. A magnet can be placed in close proximity with the assay dish in order prevent loss of immobilized antibodies during washing steps. All combinations of antibody pairs are tested to identify ones most sensitive to CRISP-3. For visualization, a standard secondary antibody linked to alkaline phosphatase (AP) is used for chemiluminescent detection.

### Example 12

Comparison of Results from Immunoassays and in situ Hybridizations of Paraffin Fixed and Frozen Tissues of Prostatectomy Specimens

Levels of CRISP-3 expression are determined by performing immunoassays to detect CRISP-3 polypeptides or by performing in situ hybridization to measure the levels of CRISP-3 mRNAs. An immunoassay of a prostate tissue specimen has the advantage of providing both a CRISP-3 polypeptide expression pattern and details regarding zonal localization of cancer within the prostate. Since CRISP-3 is a secreted protein, an elevated level of CRISP expression may not be detectable by immune histochemical assay as only CRISP-3 polypeptides retained in the cells at the time of fixation are detected in the immunoassay. To determine whether results of immunoassays for CRISP-3 polypeptides correlate with results obtained from in situ hybridizations of CRISP-3 mRNAs, frozen tissue sections and paraffin embedded tissues are analyzed by both methods. Adjacent tissue slices are examined for CRISP-3 polypeptides using a CRISP-3 specific antibody and by in situ hybridization for CRISP-3 mRNA using a CRISP-3 ribo-probe described in Example 6. Results from both methods are compared.

### Example 13

Elevated Levels of CRISP-3 mRNA were Detected in Cancerous Prostate Epithelial Cells of Various Gleason Scores

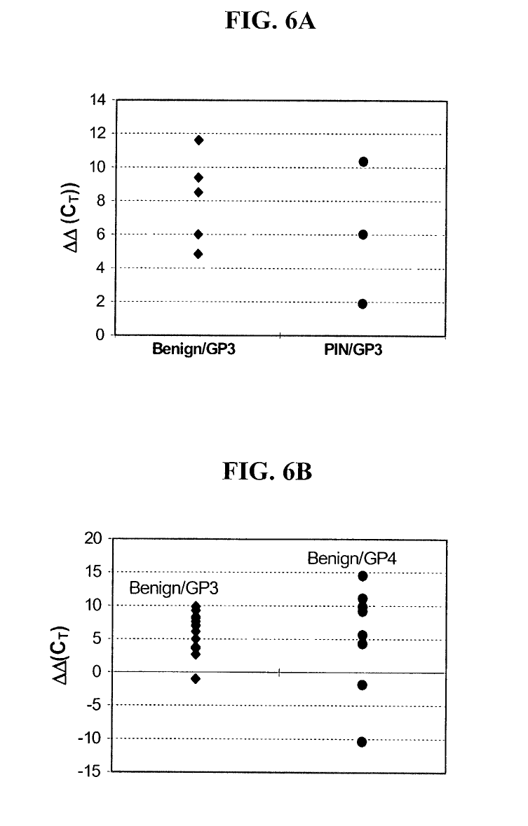

Real time RT-PCR was used to compare the level of CRISP-3 mRNA in Gleason score 6 prostate adenocarcinoma (GP3) with that in benign prostate epithelial cells (benign) and in high-grade prostate intraepithelial neoplasia (PIN). Primer sequences and PCR conditions are as described in Example 4. C_{T} values determined from real time RT-PCR were normalized using the C_{T} value for GAPDH. The difference between the C_{T} values of two samples being compared is represented by ΔΔ(C_{T}). Five benign/GP3 and three PIN/GP3 comparisons were made. FIG. 6a illustrates ΔΔ(C_{T}) results. Levels of CRISP-3 mRNA were significantly elevated in GP3 (Gleason score 6) adenocarcinoma compared to those in the benign sample. In these comparisons, ΔΔ(C_{T}) values ranged from 5 to 9. A difference in the level of CRISP-3 mRNA also was found between GP3 (Gleason score 6) prostate adenocarcinoma and PIN. ΔΔ(C_{T}) values from 2 to 10 were observed when GP3 and PIN samples were compared.

Levels of CRISP-3 mRNA in bulk samples of moderately differentiated (GP3: Gleason score 6, n=15) and poorly differentiated (GP4: Gleason scores 8 and greater, n=8) prostate adenocarcinoma (FIG. 6b) also were compared with that in benign prostate cells as described above. In 20 of 23 cases, elevated levels of CRISP-3 mRNA were observed in the adenocarcinoma samples compared to benign samples. In one case, the level of CRISP-3 mRNA was suppressed in the poorly differentiated adenocarcinoma relative to that in the benign sample. This experiment was repeated two additional times using LCM and identical results were obtained.

### Example 14

Correlating CRISP-3 Expression in Histological Specimens with Cancer Aggressiveness and Clinical Outcome

The stage and aggressiveness of prostate cancer are determined from examining histologic samples. Prostate cancers having high Gleason scores, >=7, indicate an aggressive tumor, whereas low Gleason scores, <=5, indicate a slower growing tumor. For cancel having a Gleason score of 6, however, a reliable prognosis cannot be made. To show that CRISP-3 is a useful marker for determining aggressiveness of prostate cancer, the levels of CRISP-3 expression in Prostatectomy specimens known clinical outcomes are analyzed. In this way, a CRISP-3 expression profile that can be used in determining the prognosis of any prostate cancer case based on CRISP-3 expression level is generated.

Frozen prostate tissue sections and paraffin embedded tissues representing normal, BPN, and different Gleason grade cancers are used in generating a CRISP-3 expression profile. Specimen samples having Gleason scores of 6 or 7 are of special interest, as reliable prognosis of tumor progression cannot be made with certainty in these cases. The level of CRISP-3 expression is determined for each sample. Since the patient case history corresponding to each sample is known, the level of CRISP-3 expression is correlated with progression stage and clinical outcome. In each case, PSA staining can be used as reference and positive control.

Methods for determining CRISP-3 mRNA and polypeptide expression levels in these samples include real time RT-PCR, in situ hybridization, and immunoassays. These techniques are performed as described above. For real time RT-PCR, mRNA from frozen tissues is used.

In this way, levels of CRISP-3 mRNA and polypeptide expression ale measured to determine a correlation with cancer prognosis and patient outcome.

### Example 15

Correlating Blood Levels of CRISP-3 Polypeptides with Blood Levels of PSA and Cancer Progression

To determine whether the levels of CRISP-3 polypeptides in the blood can be used to diagnose prostate cancer, levels of CRISP-3 polypeptides in the blood of prostate cancer patients are measured and compared with PSA levels in the same samples.

Blood samples can be obtained from clinical waste samples that are used for PSA tests. Fifty blood samples with PSA values ranging from a normal ˜2 ng/mL to high >80 ng/mL are used. For each sample, CRISP-3 and PSA levels are measured using ELISA. PSA levels are determined using the DAKO Envision System (DAKO Corp). CRISP-3 levels also are determined using the same DAKO Envision System and an anti-CRISP-3 antibody. CRISP-3 levels are determined using the affinity antibody described in Example 9 or 10 as the capture antibody in ELISA. A second antibody that recognizes a second epitope on the CRISP-3 polypeptide will be used as the detection antibody.

In this way, the blood levels of CRISP-3 and PSA polypeptides are used to generate a correlation plot illustrating blood levels of CRISP-3 and PSA polypeptides with cancer progression. Some correlation between blood levels of CRISP-3 and blood levels of PSA polypeptides are expected as blood levels of both PSA and CRISP-3 polypeptides increase in the blood of cancer patients. The correlation between blood levels of CRISP-3 with cancer progression is expected to be more useful for cancer diagnosis and prognosis as levels of CRISP-3 mRNA and polypeptide expression is elevated in cancerous cells.

### Example 16

Examination of CRISP-3 mRNA Expression in Response to Hormones

To determine if androgen and glucocorticoid affect the level of CRISP-3 mRNA expression, levels of CRISP-3 mRNA expression are examined in both cancerous and benign prostate cells.

Cell lines used in the analysis include the Prostate benign cell line BPH1 and the prostate cancer cell lines PC-3, DU145, and LNCaP. Cells are cultured in the presence and absence of various concentrations of testosterone and dexamethasone (DEX). For each cell line, duplicate samples with equal numbers of cells are cultured. At 48 hours after incubation, the level of CRISP-3 mRNA expression in one sample of each cell line is determined by real-time RT-PCR. This result represents the level of CRISP-3 mRNA expression in the presence of androgen and steroid. At the same time, the other sample is transferred to culture medium containing no hormone or steroid and incubated for another 24 hours. After the second incubation period, the level of CRISP-3 mRNA expression is again measured by real time RT-PCR. Incubation in culture medium containing no hormone or steroid allows for determining the effect of hormone withdrawal on the level of CRISP-3 mRNA expression. Similar levels of CRISP-3 mRNA expression in the presence and absence of hormones indicate that the level of CRISP-3 mRNA expression is not regulated by hormones. Therefore the level of CRISP-3 mRNA expression is likely to be similar among different individuals and a basal level of expression in normal individuals can be established. Furthermore, this similarity also indicates that the level of CRISP-3 mRNA expression is unlikely to vary in a patient undergoing hormonal therapy or Prostatectomy. In contrast, any significant difference in the levels of CRISP-3 mRNA expression in the presence and absence of hormones indicates that basal levels of CRISP-3 expression among normal individuals are different and further that hormonal therapy or prostatectomy likely induces changes in the level of CRISP-3 expression. As described in previous examples, the levels of GAPDH and PSA mRNA expression are used as controls in this experiment.

### Example 17

Chances in CRISP-3 mRNA Expression in Other Non Prostate Tissues in Response to Cancer

In humans, CRISP-3 is expressed in cells of the pancreas, salivary gland, thymus, ovary, testis, and descending colon in addition to cells of the prostate. Computational data from Example 7 indicated that CRISP-3 also is expressed in blood (CML). To determine if CRISP-3 can be a diagnostic and or prognostic marker for cancer in these types of tissues, the relative levels of CRISP-3 mRNA expression in cancerous and normal cells from these tissues are determined.

The levels of CRISP-3 mRNA expression are examined by real time RT-PCR and by in situ hybridization techniques as described in Example 13. The levels of CRISP-3 mRNA expression at various stages of cancer progression in the different tissues also are examined using High-Throughput Tissue Microarray Analysis. Immunohistochemistry using either CRISP-3 polyclonal or monoclonal antibodies also are used to analyze the levels of CRISP-3 polypeptide expression. Any changes in the levels of CRISP-3 expression due to cancer are examined.

Differences in the levels of CRISP-3 mRNA and polypeptide expression in cancerous and normal cells from each tissue type are used for tissue specific cancer diagnosis and prognosis. Comparisons of the levels of CRISP-3 mRNA and polypeptide expression in cells from different tissues are useful for determining if CRISP-3 expression patterns can distinguish prostate cancer from other forms of cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether or not a patient has cancer comprising detecting CRISP-3 expression in a biological sample from said patient.

2. The method of claim 1, further comprising determining the level of CRISP-3 expression in said biological sample from said patient.

3. The method of claim 2, wherein said level of CRISP-3 expression is determined by measuring the level of CRISP-3 RNA or CRISP-3 polypeptide.

4. The method of claim 1, wherein said patient is a mammal.

5. The method of claim 4, wherein said mammal is a human.

6. The method of claim 1, wherein said cancer is prostate cancer.

7. The method of claim 1, wherein said cancer is pancreas cancer.

8. The method of claim 1, wherein said cancer is salivary gland cancer.

9. The method of claim 1, wherein the cancer is lung cancer.

10. The method of claim 1, wherein the cancer is ovary cancer.

11. The method of claim 1, wherein the cancer is thymus cancer.

12. The method of claim 1, wherein the cancer is hematological cancer

13. The method of claim 1, wherein the cancer is descending colon cancer.

14. The method of claim 1, wherein said biological sample is a histological specimen.

15. The method of claim 14, wherein said histological specimen is a prostate epithelial cell specimen.

16. The method of claim 14, wherein said histological specimen is a pancreas cell specimen.

17. The method of claim 14, wherein said histological specimen is a specimen of a metastatic tumor.

18. The method of claim 1, wherein said biological sample comprises a body fluid.

19. The method of claim 18, wherein said body fluid comprises blood.

20. The method of claim 18, wherein said body fluid comprises bone marrow.

21. The method of claim 18, wherein said body fluid comprises urine.

22. The method of claim 18, wherein said body fluid comprises semen.

23. The method of claim 1, wherein said body fluid comprises saliva.

24. The method of claim 18, wherein said body fluid comprises vaginal secretions.

25. The method of claim 18, wherein said body fluid comprises cerebrospinal fluid.

26. The method of claim 18, wherein said body fluid comprises pancreatic fluid.

27. A method for identifying a cancer cell in a biological sample, said method comprising detecting CRISP-3 RNA or CRISP-3 polypeptide in said biological sample.

28. The method of claim 27, further comprising determining the level of said CRISP-3 RNA or CRISP-3 polypeptide in said biological sample.

29. A method for distinguishing benign prostate hyperplasia, prostate intraepithelial neoplasia, and cancer, said method comprising determining the level of CRISP-3 RNA or CRISP-3 polypeptide in a biological sample and correlating said level of CRISP-3 RNA or CRISP-3 polypeptide in said biological sample with BPH or PIN.

30. A method for determining a prognosis for a cancer condition comprising determining the level CRISP-3 RNA or CRISP-3 polypeptide in a biological sample and correlating said level of said CRISP-3 RNA or CRISP-3 polypeptide with a cancer prognosis.

31. A purified antibody that binds specifically to a CRISP-3 polypeptide.

32. A method of making a CRISP-3 antibody, said method comprising immunizing a non-human animal with a CRISP-3 polypeptide and isolating the serum of said immunized non-human animal.

33. The method of claim 32, further comprising isolating from said serum of said immunized non-human animal, an antibody that will specifically bind to said CRISP-3 polypeptide.

34. A method of making a CRISP-3 antibody, said method comprising immunizing a non-human animal with a nucleic acid molecule that allows for expression of a CRISP-3 polypeptide in said non-human animal and isolating the serum of said immunized non-human animal.

35. The method of claim 34, further comprising isolating from said serum of said immunized non-human animal, an antibody that will specifically bind to said CRISP-3 polypeptide.

36. The method of claim 32 or 34, wherein said non-human animal is selected from the group consisting of a rabbit, a chicken, a mouse, a guinea pig, a rat, a sheep, and a goat.

37. A method of making a CRISP-3 antibody, said method comprising providing a hybridoma cell that produces a monoclonal antibody specific for said CRISP-3 polypeptide, culturing said hybridoma cell under conditions that permit production of said monoclonal antibody, and isolating said monoclonal antibody from the culture supernatant of said hybridoma cell.

38. A method for determining the presence or absence of a cancer cell in a biological sample comprising contacting said biological sample with a CRISP-3 antibody and detecting the presence or absence of specific hybridization of said CRISP-3 antibody with said cancer cell.

39. An isolated nucleic acid of 800 bases or less that will hybridize under high stringency conditions to a nucleic acid consisting of nucleotides 650 to 1450, or the complement thereof, of SEQ ID NO: 1.

40. An isolated nucleic acid that will hybridize under high stringency conditions to the 3′ untranslated region of human CRISP-3 RNA but not to the 3′ untranslated regions of human CRISP-1 and CRISP-2 RNA.

41. An isolated nucleic acid consisting essentially of a sequence selected from the group consisting of SEQ ID NO: 6, SEQ ID NO: 7, SEQ ID NO: 12, SEQ ID NO: 13, SEQ ID NO: 16, SEQ ID NO: 17, SEQ ID NO: 18, SEQ ID NO: 19, and SEQ ID NO: 20.

42. The isolated nucleic acid of claim 39 or 41 wherein said isolated nucleic acid is labeled.

43. A method for determining the presence or absence of a cancer cell in a biological sample comprising contacting said biological sample with the isolated nucleic acid of claim 39 and detecting the presence or absence of specific hybridization of said isolated nucleic acid with said cancer cell.

44. A method for determining the presence or absence of a cancer cell in a biological sample, said method comprising:
(a) isolating RNA from said biological sample,
(b) performing a nucleic acid amplification reaction using a CRISP-3 primer, and
(c) detecting the presence or absence of a CRISP-3 nucleic acid amplification product in said amplification reaction.

45. The method of claim 44, wherein said nucleic acid amplification is performed using RT-PCR.

46. A kit comprising the antibody of claim 31.

47. The kit of claim 46, further comprising a PSA antibody.

48. A kit comprising the nucleic acid of claim 39.

49. The kit of claim 48, further comprising a nucleic acid that will hybridize under high or moderate stringency conditions with a PSA RNA.
